(19) United States
(12) Patent Application Publication
(10) Pub. No.: US 2025/0333449 A1
(43) Pub. Date: Oct. 30, 2025

(54) SCALABLE FERMENTATION PROCESS

(71) Applicant: KUROS BIOSCIENCES AG, Schlieren (CH)

(72) Inventors: Marcel EMMERLING, Schlieren (CH); Frank HENNECKE, Dietlikon (CH); Holger PFRÜNDER, Zürich (CH); Martin RHIEL, Bonstetten (CH); Philipp STEINER, Schlieren (CH)

(21) Appl. No.: 18/813,941

(22) Filed: Aug. 23, 2024

Related U.S. Application Data
(63) Continuation of application No. 17/229,355, filed on Apr. 13, 2021, now abandoned.
Application No. 17/229,355 is a continuation of application No. 16/654,232, filed on Oct. 16, 2019, now abandoned.
Application No. 16/654,232 is a continuation of application No. 15/353,884, filed on Nov. 17, 2016, now abandoned.
Application No. 15/353,884 is a continuation of application No. 14/247,097, filed on Apr. 7, 2014, now Pat. No. 9,518,095.
Application No. 14/247,097 is a continuation of application No. 13/335,008, filed on Dec. 22, 2011, now abandoned.
Application No. 13/335,008 is a continuation of application No. 11/921,023, filed on Nov. 26, 2007, now abandoned.

(30) Foreign Application Priority Data
May 26, 2005 (EP) 05011416.4
Jul. 21, 2005 (EP) 05106729.6

(51) Int. Cl. C07K 14/005; A61K 39/00; C12N 7/00

(52) U.S. Cl. CPC C07K 14/005; C12N 7/00; A61K 2039/5256; A61K 2039/5258; C12N 2795/00023; C12N 2795/00051; C12N 2795/10051; C12N 2795/10061; C12N 2795/18022; C12N 2795/18052

(57) ABSTRACT

This invention provides a robust fermentation process for the expression of a capsid protein of a bacteriophage which is forming a VLP by self-assembly, wherein the process is scalable to a commercial production scale and wherein the expression rate of the capsid protein is controlled to obtain improved yield of soluble capsid protein. This is achieved by combining the advantages of fed-batch culture and of lactose induced expression systems with specific process parameters providing improved repression of the promoter during the growth phase and high plasmid retention throughout the process.

### SEQUENCE LISTING

The instant application includes a Sequence Listing which has been submitted electronically in XML format and is hereby incorporated by reference in its entirety. The Sequence Listing, created on Mar. 26, 2020, is named “0156-0003US5_SL” and is 74,064 bytes in size.

### FIELD OF THE INVENTION

This invention is related to the field of protein expression and fermentation technology. A process for the efficient expression of recombinant bacteriophage capsid protein in a bacterial host is described. The process leads to high yield of recombinant capsid protein which is capable of forming a virus-like particle (VLP) by self-assembly. Furthermore, the process is scalable from laboratory scale to fermenter volumes larger than 50 litres.

### BACKGROUND OF THE INVENTION

Recent vaccination strategies make use of viruses or virus-like-particles (VLPs) to enhance the immune response towards antigens. For example, WO02/056905 demonstrates the utility of VLPs as a carrier to present antigens linked thereto in a highly ordered repetitive array. Such antigen arrays can cause a strong immune response, in particular antibody responses, against the linked antigen and are even capable of breaking the immune system's inherent tolerance towards self antigens. Such antigen arrays are therefore useful in the production of vaccines for the treatment of infectious diseases and allergies as well as for the efficient induction of self-specific immune responses, e.g. for the treatment of cancer, rheumatoid arthritis and various other diseases.

As indicated in WO02/056905 capsid proteins of bacteriophages are particularly suited as antigen carrier. They have been shown to efficiently self-assemble into VLPs upon expression in a bacterial host (Kastelein et al. 1983, Gene 23:245-254; Kozlovskaya et al. 1986, Dokl. Akad. Nauk SSSR 287:452-455). Moreover, capsid proteins of bacteriophages such as derived from fr (Pushko et al. 1993, Protein Engineering 6(8)883-891), Qβ (Kozlovska et al. 1993, Gene 137:133-137; Ciliens et al. 2000, FEBS Letters 24171:1-4; Vasiljeva et al 1998, FEBS Letters 431:7-11) and MS-2 (WO92/13081; Mastico et al. 1993, Journal of General Virology 74:541-548; Heal et al. 2000, Vaccine 18:251-258) have been produced in bacterial hosts using inducible promoters such as the trp promoter or a trβ-T7 fusion (in the case of fr and Qb) or the tac promoter using IPTG as inducer substance (in the case of MS-2). The use of inducible promoters is beneficial, to avoid possible toxic effects of the recombinant capsid protein and the metabolic burden of protein expression which both might reduce the growth of the bacterial expression host and, ultimately, the yield of expressed protein.

However, the expression systems used so far for the expression of capsid proteins of bacteriophages have been applied in small scale fermentations, i.e. in laboratory scale and small batch cultures with volumes of typically clearly below 1 litre. An scale up of these systems comprising volumes of 50 litre and more is expected to diminish in a great extent the respective capsid protein yield due to increased promoter leakage and/or lowered plasmid retention.

A further problem associated with commercially desired high-level expression and rapid accumulation of recombinant capsid proteins of bacteriophages is the formation of incorrectly folded protein species and the formation of so called inclusion bodies, i.e. protein aggregates, which are insoluble and which may hamper further downstream processes. Thus, for bacteriophage MS-2 coat protein the formation of protein aggregates and of protein species which lost their ability to self-assemble to VLPs have been reported when the protein was expressed under the control of the strong T7 promoter after IPTG induction using the pET expression system (Peabody & Al-Bitar 2001, Nucleic Acid Research 29(22):e113).

High expression rates of the recombinant capsid protein may therefore have a negative impact on the yield of correctly assembled VLPs. The production of VLP-based vaccines in a commercial scale requires, therefore, the establishment of an efficient, and in particular scalable fermentation process for the expression of recombinant capsid protein of bacteriophages leading to a product of constant quality and purity having the capability of self-assembling into VLPs, whereby the formation of insoluble fractions of the capsid protein is minimised or avoided.

Therefore, it is an object of the present invention to provide a process for expression of a recombinant capsid protein of a bacteriophage which avoids or minimizes the disadvantage or disadvantages of the prior art processes, and in particular, which is scalable to a commercial scale and still leading to a product of constant quality and purity and the capability of self-assemblance to VLPs, and wherein the formation of insoluble fraction of the capsid protein is minimised or avoided.

### SUMMARY OF THE INVENTION

The invention relates to a process for expression of a recombinant capsid protein of a bacteriophage, or a mutant or fragment thereof being capable of forming a VLP by self-assembly, said process comprising the steps of: a.) introducing an expression plasmid into a bacterial host, wherein said expression plasmid comprises an expression construct, wherein said expression construct comprises (i) a first nucleotide sequence encoding said recombinant capsid protein, or mutant or fragment thereof, and (ii) a promoter being inducible by lactose; b.) cultivating said bacterial host in a medium comprising a major carbon source; wherein said cultivating is performed in batch culture and under conditions under which said promoter is repressed by lacI, wherein said lacI is overexpressed by said bacterial host; c.) feeding said batch culture with said major carbon source; and d.) inducing said promoter with an inducer, wherein preferably said feeding of said batch culture with said major carbon source is continued.

This invention provides a robust fermentation process for the expression of a capsid protein of a bacteriophage which is forming a VLP by self-assembly, wherein the process is scalable to a commercial production scale and wherein the expression rate of the capsid protein leads to improved yield of soluble capsid protein. This is, in particular, achieved by improved repression of the promoter during the growth phase and high plasmid retention throughout the process. The expression system further avoids formation of insoluble protein aggregates by limiting the maximum expression rate occurring during the production phase.

In a preferred embodiment said bacteriophage is a RNA bacteriophage. More preferably, said RNA bacteriophage is selected from the group consisting of: a.) bacteriophage Qβ; b.) bacteriophage AP205; c.) bacteriophage fr; d.) bacteriophage GA; e.) bacteriophage SP; f.) bacteriophage MS2; g.) bacteriophage M11; h.) bacteriophage MX1; i.) bacteriophage NL95; j.) bacteriophage f2; k.) bacteriophage PP7 and l.) bacteriophage R17. Preferably, said RNA bacteriophage is Qβ. More preferably said recombinant capsid protein comprises or alternatively consists of an amino acid sequence selected from the group consisting of SEQ ID NO:5, SEQ ID NO:7, SEQ ID NO:8, SEQ ID NO:9, SEQ ID NO:10, and SEQ ID NO:11. Still more preferably said recombinant capsid protein comprises SEQ ID NO:5, most preferably said recombinant capsid protein consists of SEQ ID NO:5.

In a further preferred embodiment said recombinant capsid protein comprises or alternatively consists of an amino acid sequence selected from the group consisting of SEQ ID NO:12, SEQ ID NO:13, and SEQ ID NO:14. More preferably said recombinant capsid protein comprises SEQ ID NO:12, most preferably said recombinant capsid protein consists of SEQ ID NO:12.

In another embodiment of the present invention, said expression construct comprises a first stop codon, and wherein said first stop codon is TAA, and wherein preferably said TAA is located directly 3′ of said first nucleotide sequence.

In a further embodiment said expression construct comprises a first stop codon and a second stop codon, wherein said first stop codon is located directly 3′ of said first nucleotide sequence and wherein said second stop codon is located directly 3′ of said first stop codon, and wherein at least one of said first or second stop codon is TAA.

In a further embodiment said expression construct comprises a first nucleotide sequence and a second nucleotide sequence, wherein said first nucleotide sequence is encoding a recombinant capsid protein, preferably Qβ CP, or a mutant or fragment thereof, and wherein said second nucleotide sequence is encoding any other protein, preferably the Qβ A1 protein or a mutant or fragment thereof, and wherein said first and said second nucleotide sequence are separated by exactly one sequence stretch comprising at least one TAA stop codon. In a preferred embodiment said expression construct comprises or alternatively consists of the nucleotide sequence of SEQ ID NO:6.

In a further embodiment said expression plasmid comprises or, more preferably, consists of the nucleotide sequence of SEQ ID NO:1.

In one embodiment of the invention said promoter is selected from the group consisting of the a.) tac promoter; b.) trc promoter; c.) tic promoter; d.) lac promoter; e.) lacUV5 promoter; f.) P_{syn} promoter; g.) lpp^{a} promoter; h.) lpp-lac romoter; i.) T7-lac promoter; j.) T3-lac promoter; k.) T5-lac promoter; and l.) a promoter having at least 50% sequence homology to SEQ ID NO:2. In a preferred embodiment said promoter has at least 50%, 60%, 70%, 80, 90, or 95%, preferably 98 to 100%, most preferably 99% sequence homology to SEQ ID NO:2. In a further preferred embodiment said promoter is selected from the group consisting of tic promoter, trc promoter and tac promoter. Even more preferably said promoter is the tac promoter. Most preferably said promoter comprises or alternatively consists of the nucleotide sequence of SEQ ID NO:2.

In one embodiment said major carbon source is glucose or glycerol, preferably glycerol.

In one embodiment said feeding of said batch culture is performed with a flow rate, wherein said flow rate increases with an exponential coefficient μ, and wherein preferably said exponential coefficient μ is below μₘₐₓ.

In a further embodiment said inducing of said promoter is performed by co-feeding said batch culture with said inducer, preferably lactose and said major carbon source, preferably glycerol, at a constant flow rate.

In a further embodiment said inducing of said promoter is performed by co-feeding said batch culture with said inducer, preferably lactose and said major carbon source, preferably glycerol, at an increasing flow rate.

In a further embodiment said inducer is lactose, wherein preferably said lactose and said major carbon source are co-fed to said batch culture in a ratio of about 2:1 to 1:4 (w/w).

In a further embodiment said inducer is IPTG wherein preferably the concentration of said IPTG said medium is 0.001 to 5 mM, preferably 0.001 to 1 mM, more preferably 0.005 to 1 mM, still more preferably 0.005 to 0.5 mM. In a very preferred embodiment said concentration of IPTG is about 0.01 mM, most preferably 0.01 mM.

In one embodiment said lacI is overexpressed by said bacterial host, wherein said overexpression is caused by lacI^{q} or lacQ1, preferably by lacI^{q}. In one embodiment said bacterial host comprises said lacI^{q} gene or said lacQ1 gene, preferably said lacI^{q} gene on its chromosome. In a further preferred embodiment said bacterial host comprises said lacI^{q} gene or said lacQ1 gene, preferably said lacI^{q} gene on a plasmid, preferably on a high copy number plasmid. In a further preferred embodiment said bacterial host comprises said lacI^{q} gene or said lacQ1 gene, preferably said lacI^{q} gene on said expression plasmid.

In one embodiment said bacterial host is selected from the group consisting of the strains E. coli RB791, E. coli DH20 and E. coli Y1088. Preferably said bacterial host is E. coli RB791.

In one embodiment said bacterial host comprises β-galactosidase activity.

In one embodiment said cultivating and said feeding of said batch culture and said inducing of said promoter is performed at a temperature which is below the optimal growth temperature of said bacterial host. Preferably said temperature is between 23° C. and 35° C., more preferably between 25 and 33° C., even more preferably between 27 and 32° C., still more preferably between 28 and 31° C. Even more preferably said temperature is about 30° C., most preferably said temperature is 30° C.

In one embodiment said cultivating and said feeding of said batch culture is performed at a temperature which is below the optimal growth temperature of said bacterial host, wherein preferably said temperature is between 23° C. and 35° C., more preferably between 25 and 33° C., even more preferably between 27 and 32° C., still more preferably between 28 and 31° C., even more preferably said temperature is about 30° C., most preferably said temperature is 30° C., and said inducing of said promoter is performed at the optimal growth temperature of the bacterial host, preferably at about 37° C.

In one embodiment said cultivating and said feeding of said batch culture and said inducing of said promoter is performed in the absence of an antibiotic.

In a specific embodiment said expression plasmid comprises or alternatively consists of the nucleotide sequence of SEQ ID NO:1, said major carbon source is glycerol, said feeding of said batch culture is performed with a flow rate, wherein said flow rate increases with an exponential coefficient μ, and wherein said exponential coefficient μ is below μₘₐₓ, said inducing of said promoter by co-feeding said batch culture is performed with a constant flow rate, wherein lactose and glycerol are co-fed to the batch culture in a ratio of about 2:1 to about 1:4 (w/w), preferably about 1:1 to about 1:4 (w/w), most preferably about 1:3 (w/w), and wherein said cultivating and feeding of said batch culture and said inducing of said promoter is performed at a temperature between 27 and 32° C., preferably about 30° C., most preferably 30° C.

In a further specific embodiment said expression plasmid comprises or alternatively consists of the nucleotide sequence of SEQ ID NO:30, said major carbon source is glycerol, said feeding of said batch culture is performed with a flow rate, wherein said flow rate increases with an exponential coefficient μ, and wherein said exponential coefficient μ is below μₘₐₓ, said inducing of said promoter by co-feeding said batch culture is performed with a constant flow rate, wherein lactose and said major carbon source are co-fed to the batch culture in a ratio of about 2:1 to about 1:4 (w/w), preferably about 1:1 to about 1:4 (w/w), most preferably about 1:3 (w/w), and wherein said cultivating and feeding of said batch culture and said inducing of said promoter is performed at a temperature between 27 and 32° C., preferably about 30° C., most preferably 30° C.

### DESCRIPTION OF THE FIGURES

FIG. 1: Fermentation profile with pTac-nSD-Qb-mut (SEQ ID NO:1) in RB791 in 21 culture. Co-feeding during production phase was performed with medium containing 20% glycerol and 20% lactose. Shown are glycerol concentration [g/l](circles); lactose concentration [g/l](triangles); β-Gal activity [U/ml*OD=1](squares) and OD600 (diamonds) plotted against the process time [h].

### DETAILED DESCRIPTION OF THE INVENTION

Unless defined otherwise, all technical and scientific terms used herein have the same meanings as commonly understood by one of ordinary skill in the art to which this invention belongs.

“about”: within the meaning of the present application the expression about shall have the meaning of +/−10%. For example about 100 shall mean 90 to 110.

“promoter which is inducible by lactose” as used herein refers to a promoter which comprises regulatory elements of the lac operon. Such promoters are repressed by lacI and can be induced by lactose or the synthetic inducer IPTG. The skilled person is aware that induction of a promoter by lactose requires β-galactosidase activity in the bacterial host.

“located directly 3′”: a nucleotide sequence N2 which is located directly 3′ of another nucleotide sequence N1 refers to a continuous sequence having the conformation 5′-N1-N2-3′ wherein N1 and N2 are directly connected and not separated by additional sequence elements.

“sequence stretch”: as used herein the term “sequence stretch” refers to a continuous nucleotide sequence which consists of less than 50, preferably less than 20, more preferably less than 10, even more preferably less than 5 nucleotides. In a further preferred embodiment the sequence stretch comprises or alternatively consists of at least one, preferably one, TAA stop codon. In another embodiment the sequence stretch comprises or alternatively consists of at least one, preferably one, TAA and at least one, preferably one, TGA stop codon. In further preferred embodiment the sequence stretch comprises or alternatively consists of SEQ ID NO:32.

“bacterial host”: as used herein the term “bacterial host” refers to a bacterial organism which is hosting or capable of hosting an expression plasmid of the invention, wherein “hosting” involves the replication of the expression plasmid and maintenance of the expression plasmid during cell division.

“culture”: in the context of the instant invention a “culture” comprises a bacterial host in a medium (“bacterial culture”), wherein typically said medium is supporting the growth of said bacterial host.

“batch culture” as used herein relates to a culture, i.e. a bacterial host in a medium, wherein said culture constitutes a closed system, i.e. typically and preferably no addition or removal of medium takes place during the cultivation time. Therefore, in contrast to a continuous culture, typically and preferably the density of the bacterial host in the batch culture continuously increases with progressing cultivation time. Batch culture does not exclude the addition of compounds required for the control of the process, such as, for example, inducer, oxygen, and alkali or acid to control the pH.

“fed batch culture”: as used herein is a culture which is supplied with additional medium comprising a substrate, preferably the major carbon source of the bacterial host (feed or co-feed medium). In the context of the application this process is referred to by the terms “feeding said batch culture” (medium comprises the major carbon source) and “co-feeding said batch culture” (medium comprises the major carbon source and the inducer, preferably lactose). Typically and preferably, no removal of medium except for analytical purposes takes place during cultivation time of a fed batch culture.

“Preculture”: a culture, preferably a batch culture, which is used to produce the inoculum for a culture of a larger volume, e.g. the culture in which the recombinant capsid protein is produced (production culture). A preculture can be performed in two or more steps, wherein a second preculture is inoculated with a first preculture etc. to produce a sufficiently large inoculum for the production culture. The first and/or subsequent precultures may comprise an antibiotic to improve plasmid stability.

“substrate”: as used herein refers to a compound in the culture medium which contributes to the carbon and energy supply of the bacterial host. The terms “substrate” therefore encompasses any compound contained in the medium contributing to the carbon supply of the bacterial host. Typical substrates for bacteria are sugar, starch, glycerol, acetate and any other organic compound which can be metabolized by bacteria. Therefore, the term “substrate” includes the major carbon source but also, for example, lactose.

“Major carbon source” as used herein refers to the compound in the culture medium which contributes most to the carbon and energy supply of the bacterial host during the growth phase. The major carbon source thus is the major substrate of the bacterial host. The major carbon source is typically a sugar such as sucrose or glucose, or glycerol, and preferably glucose or glycerol. Though lactose could in principal act as a major carbon source for a bacterial host, in the context of the instant invention the term “major carbon source” typically and preferably does not include lactose.

Phases of the process of the invention: The process of the invention is characterised by different phases which refer to different physiological conditions of the bacterial host with respect to its growth and the repression/induction status of the expression construct.

“Growth phase”: The growth phase is initiated by said cultivating said bacterial host in a medium. The growth phase is preferably characterized by conditions under which the promoter driving the expression of the recombinant capsid protein is repressed and the growth phase is terminated with said inducing said promoter with an inducer. The growth phase can be further divided in a “batch phase” and a “feed phase”. Said batch phase is initiated by said cultivating said bacterial host in a medium. The batch phase comprised a “lag phase” during which the bacterial host is not yet growing or growing with a non-exponential rate, typically and preferably a linear rate. The growth phase further comprises an “exponential growth phase” which directly follows the lag phase. No feeding of said culture takes place during the batch phase, thus the exponential growth phase is terminated by the consumption of the substrate by the bacterial host. The growth phase further comprises a “feed phase” which is directly following the batch phase and which is initiated by said feeding of said batch culture with said major carbon source. The feed phase is characterised by a growth rate of the bacterial host which is directly dependent on the flow rate of the feed medium containing the major carbon source.

“production phase”: The growth phase is followed by the production phase which is initiated by said inducing said promoter with an inducer, wherein typically and preferably said feeding of said batch culture with said major carbon source is continued.

“Conditions under which the promoter is repressed”: it is to be understood that the repression of a promoter is an equilibrium of formation and dissociation of the repressor-operator complex and that even stringently repressed promoters may show a certain expression rate also in the absence of their inducer. Therefore, as used within the application the term “conditions under which the promoter is repressed” relates to conditions, wherein at the end of the growth phase, i.e. directly before the addition of inducer to the culture, the recombinant capsid protein is expressed to a level which does not exceed a concentration in the medium of 200 mg/l, preferably 150 mg/l, more preferably 100 mg/l, as determined by the HLPC method of Example 17. Most preferably, the concentration of the recombinant protein is below the detection level of said method.

“Inducer”: within the meaning of the in invention the term “inducer” relates to any substance which directly or indirectly interacts with an inducible promoter and thereby facilitates expression from said promoter; for example, inducers of “a promoter inducible by lactose”, such as the lac or tac promoter, are IPTG, lactose and allolactose.

“Coat protein”/“capsid protein”: The term “coat protein” and the interchangeably used term “capsid protein” within this application, refers to a viral protein, preferably a subunit of a natural capsid of a virus, preferably of a RNA bacteriophage, which is capable of being incorporated into a virus capsid or a VLP. For example, the specific gene product of the coat protein gene of RNA bacteriophage Qβ is referred to as “Qβ CP”, whereas the “coat proteins” or “capsid proteins” of bacteriophage Qβ comprise the “Qβ CP” as well as the A1 protein.

“Recombinant capsid protein”: A capsid protein which is synthesised by a recombinant host cell.

“Polypeptide”: As used herein the term “polypeptide” refers to a polymer composed of amino acid residues, generally natural amino acid residues, linked together through peptide bonds. Although a polypeptide may not necessarily be limited in size, the term polypeptide is often used in conjunction with peptide of a size of about ten to about 50 amino acids.

“Protein”: As used herein, the term protein refers to a polypeptide generally of a size of above 20, more particularly of above 50 amino acid residues. Proteins generally have a defined three dimensional structure although they do not necessarily need to, and are often referred to as folded, in opposition to peptides and polypeptides which often do not possess a defined three-dimensional structure, but rather can adopt a large number of different conformations, and are referred to as unfolded.

“Recombinant host cell”: As used herein, the term “recombinant host cell” refers to a host cell into which one ore more nucleic acid molecules of the invention have been introduced.

“Recombinant VLP”: The term “recombinant VLP”, as used herein, refers to a VLP that is obtained by a process which comprises at least one step of recombinant DNA technology. The term “VLP recombinantly produced”, as used herein, refers to a VLP that is obtained by a process which comprises at least one step of recombinant DNA technology. Thus, the terms “recombinant VLP” and “VLP recombinantly produced” are interchangeably used herein and should have the identical meaning.

“RNA-bacteriophage”: As used herein, the term “RNA-bacteriophage” refers to RNA viruses infecting bacteria, preferably to single-stranded positive-sense RNA viruses infecting bacteria.

“Virus-like particle (VLP)”: as used herein, the term “virus-like particle” refers to a structure resembling a virus particle or it refers to a non-replicative or non-infectious, preferably a non-replicative and non-infectious virus particle, or it refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. Preferably a virus-like particle in accordance with the invention is non-replicative and/or non-infectious since it lacks all or part of the viral genome or genome function. Typically a virus-like particle lacks all or part of the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, preferably RNA-phage. The terms “viral capsid” or “capsid”, refer to a macromolecular assembly composed of viral protein subunits. Typically, there are 60, 120, 180, 240, 300, 360 and more than 360 viral protein subunits. Typically and preferably, the interactions of these subunits lead to the formation of viral capsid or viral-capsid like structure with an inherent repetitive organization, wherein said structure is, typically, spherical or tubular. For example, the capsids of RNA bacteriophages or HBcAgs have a spherical form of icosahedral symmetry.

“Virus-like particle of a RNA bacteriophage”: As used herein, the term “virus-like particle of a RNA bacteriophage” refers to a virus-like particle comprising, or preferably consisting essentially of or consisting of coat proteins, mutants or fragments thereof, of a RNA bacteriophage. In addition, virus-like particle of a RNA bacteriophage resembling the structure of a RNA bacteriophage, being non replicative and/or non-infectious, and lacking at least the gene or genes encoding for the replication machinery of the RNA bacteriophage, and typically also lacking the gene or genes encoding the protein or proteins responsible for viral attachment to or entry into the host. Preferred VLPs derived from RNA bacteriophages exhibit icosahedral symmetry and consist of 180 subunits. A preferred method to render a virus-like particle of a RNA bacteriophage non replicative and/or non-infectious is by genetic manipulation.

one, a, or an: When the terms “one,” “a,” or “an” are used in this disclosure, they mean “at least one” or “one or more,” unless otherwise indicated.

“Sequence identity”: The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or a fragment thereof disclosed in this invention.

“Sequence homology”: The homology of nucleotide sequences can for example be determined by the program blastn which is an implementation of the BLAST algorithm, preferably using the default settings of the software.

“Fragment of a protein”, in particular fragment of a recombinant protein or recombinant coat protein, as used herein, is defined as a polypeptide, which is of at least 70%, preferably at least 80%, more preferably at least 90%, even more preferably at least 95% the length of the wild-type recombinant protein, or coat protein, respectively and which preferably retains the capability of forming VLP. Preferably the fragment is obtained by at least one internal deletion, at least one truncation or at least one combination thereof. Further preferably the fragment is obtained by at most 10, at most 9, at most 8, at most 7, at most 6, at most 5, at most 4, at most 3 or at most 2 internal deletions; by at most 10, at most 9, at most 8, at most 7, at most 6, at most 5, at most 4, at most 3 or at most 2 truncations; or by at most 3, preferably at most 2, most preferably by exactly one combination thereof. Most preferably the fragment is obtained by exactly one internal deletion, exactly one truncation or by a combination thereof.

The term “fragment of a recombinant protein” or “fragment of a coat protein” shall further encompass polypeptide, which has at least 80%, preferably 90%, even more preferably 95% amino acid sequence identity with the “fragment of a recombinant protein” or “fragment of a coat protein”, respectively, as defined above and which is preferably capable of assembling into a virus-like particle.

The term “mutant recombinant protein” or the term “mutant of a recombinant protein” as interchangeably used in this invention, or the term “mutant coat protein” or the term “mutant of a coat protein”, as interchangeably used in this invention, refers to a polypeptide having an amino acid sequence derived from the wild type recombinant protein, or coat protein, respectively, wherein the amino acid sequence is at least 80%, preferably at least 85%, 90%, 95%, 97%, or 99% identical to the wild type sequence and preferably retains the ability to assemble into a VLP.

The invention is related to an efficient fermentation process for the production of a VLP of a bacteriophage. The process is improved with respect to yield of the VLP and can be scaled up to a commercial production scale. The process encompasses the expression of recombinant capsid protein of bacteriophages in a bacterial host under conditions which allow the capsid protein to self-assemble into VLPs spontaneously.

Specific examples of VLPs which can be produced by the process of the invention are VLPs of bacteriophages, preferably RNA bacteriophages. In one preferred embodiment of the invention, the virus-like particle of the invention comprises, consists essentially of, or alternatively consists of, recombinant coat proteins, mutants or fragments thereof, of a RNA-phage. Preferably, the RNA-phage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; 1) bacteriophage PP7 and m) bacteriophage AP205.

In one preferred embodiment of the invention, VLPs are produced comprising coat protein, mutants or fragments thereof, of RNA bacteriophages, wherein the coat protein has an amino acid sequence selected from the group consisting of: (a) SEQ ID NO:5 □ referring to Qβ CP; (b) a mixture of SEQ ID NO:5 and SEQ ID NO:15 (Qβ A1 protein); (c) SEQ ID NO:16 (R17 capsid protein); (d) SEQ ID NO:17 (fr capsid protein); (e) SEQ ID NO:18 (GA capsid protein); (f) SEQ ID NO:19 (SP capsid protein); (g) a mixture of SEQ ID NO:19 and SEQ ID NO:20; (h) SEQ ID NO:21 (MS2 capsid protein); (i) SEQ ID NO:22 (M11 capsid protein); (i) SEQ ID NO:23 (MX1 capsid protein); (k) SEQ ID NO:24 (NL95 capsid protein); (1) SEQ ID NO:25 (f2 capsid protein); (m) SEQ ID NO:26 (PP7 capsid protein); and (n) SEQ ID NO:12 (AP205 capsid protein).

Upon expression in E. coli, the N-terminal methionine of Qβ coat protein is usually removed (Stoll, E. et al., J. Biol. Chem. 252:990-993 (1977)). VLP composed of Qβ coat proteins where the N-terminal methionine has not been removed, or VLPs comprising a mixture of Qβ coat proteins where the N-terminal methionine is either cleaved or present are also within the scope of the present invention.

In one preferred embodiment of the invention, the VLP is a mosaic VLP comprising or alternatively consisting of more than one amino acid sequence, preferably two amino acid sequences, of coat proteins, mutants or fragments thereof, of a RNA bacteriophage.

In one very preferred embodiment, the VLP comprises or alternatively consists of two different coat proteins of a RNA bacteriophage, said two coat proteins have an amino acid sequence of SEQ ID NO: 5 and SEQ ID NO:15, or of SEQ ID NO:19 and SEQ ID NO:20.

In preferred embodiments of the present invention, the produced VLP comprises, or alternatively consists essentially of, or alternatively consists of recombinant coat proteins, mutants or fragments thereof, of the RNA-bacteriophage Qβ, fr, AP205 or GA.

In one preferred embodiment, the VLP is a VLP of RNA-phage Qβ. The capsid or virus-like particle of Qβ shows an icosahedral phage-like capsid structure with a diameter of 25 nm and T=3 quasi symmetry. The capsid contains 180 copies of the coat protein, which are linked in covalent pentamers and hexamers by disulfide bridges (Golmohammadi, R. et al., Structure 4:543-5554 (1996)).

Preferred virus-like particles of RNA bacteriophages, in particular of Qβ and fr in accordance of this invention are disclosed in WO 02/056905, the disclosure of which is herewith incorporated by reference in its entirety. Particular Example 18 of WO 02/056905 gave detailed description of preparation of VLP particles from Qβ.

In another preferred embodiment, the VLP is a VLP of RNA bacteriophage AP205. Assembly-competent mutant forms of AP205 VLPs, including AP205 coat protein with the substitution of proline at amino acid 5 to threonine, may also be used in the practice of the invention and leads to other preferred embodiments of the invention. WO 2004/007538 describes, in particular in Example 1 and Example 2, how to obtain VLP comprising AP205 coat proteins, and hereby in particular the expression and the purification thereto. WO 2004/007538 is incorporated herein by way of reference.

In one preferred embodiment, the VLP comprises or consists of a mutant coat protein of a virus, preferably a RNA bacteriophage, wherein the mutant coat protein has been modified by removal of at least one lysine residue by way of substitution and/or by way of deletion. In another preferred embodiment, the VLP of the invention comprises or consists of a mutant coat protein of a virus, preferably a RNA bacteriophage, wherein the mutant coat protein has been modified by addition of at least one lysine residue by way of substitution and/or by way of insertion. The deletion, substitution or addition of at least one lysine residue allows varying the degree of coupling with an antigen.

VLPs or capsids of Q□□ coat protein display a defined number of lysine residues on their surface, with a defined topology with three lysine residues pointing towards the interior of the capsid and interacting with the RNA, and four other lysine residues exposed to the exterior of the capsid.

Qβ mutants, of which exposed lysine residues are replaced by arginines are also encompassed by the present invention. Preferably these mutant coat proteins comprise or alternatively consist of an amino acid sequence selected from the group of a) Qβ-240 (SEQ ID NO:7, Lys13□Arg); b) Qβ-243 (SEQ ID NO:8, Asn10□Lys); c) Qβ-250 (SEQ ID NO:9, Lys2□Arg); d) Qβ-251 (SEQ ID NO:10, Lys16□Arg); and e) Qβ-259 (SEQ ID NO:11, Lys2□Arg, Lys16□Arg). The construction, expression and purification of the above indicated Qβ mutant coat proteins, mutant Qβ coat protein VLPs and capsids, respectively, are described in WO02/056905. In particular is hereby referred to Example 18 of above mentioned application.

In a further preferred embodiment the recombinant capsid protein is a capsid protein of bacteriophage AP205 having the amino acid sequence depicted in SEQ ID NO:12 or a mutation thereof, which is capable of forming a VLP, for example the proteins AP205P5T (SEQ ID NO:13) or AP205 N14D (SEQ ID NO:14.).

In a very preferred embodiment said recombinant capsid protein is composed of the 133 amino acid coat protein C of E. coli RNA bacteriophage Qβ comprising or preferably consisting of the amino acid sequence depicted in SEQ ID NO:5, wherein preferably said recombinant capsid protein is capable of forming a VLP by self-assembly.

In one embodiment, the expression construct comprises a first stop codon and a second stop codon, wherein said first stop codon is located directly 3′ of said first nucleotide sequence and wherein said second stop codon is located directly 3′ of said first stop codon, and wherein at least one of said first or second stop codon is TAA. For example, plasmid pTac-nSDAP205 (SEQ ID NO:30) comprises the naturally occurring TAA stop codon as a first stop codon and an additional TGA stop codon directly 3′ of the first stop codon.

In a preferred embodiment the expression construct comprises a first nucleotide sequence and a second nucleotide sequence, wherein said first nucleotide sequence is encoding a recombinant capsid protein, preferably Qβ CP, or a mutant or fragment thereof, most preferably SEQ ID NO:5, and wherein said second nucleotide sequence is encoding any other protein, preferably the Qβ A1 protein or a mutant or fragment thereof, most preferably SEQ ID NO:15, and wherein said first and said second nucleotide sequence are separated by exactly one sequence stretch comprising at least one TAA stop codon. In one embodiment said TAA stop codon is generated by replacing the naturally occurring stop codon, preferably TGA by the sequence TAA. Alternatively and more preferrably said TAA stop codon is generated by replacing the naturally occurring stop codon, preferably TGA by the sequence TAATGA (SEQ ID NO:32).

For example, the region of Qβ gene C corresponds to the NCBI GenBank Acc. No. M99039 (nucleotides 46-1062). Gene C contains a first nucleotide sequence encoding the 133-amino acid Qβ coat protein (SEQ ID NO:5) and a second nucleotide sequence encoding the 329-amino acid read through protein A1 (SEQ ID NO:15). Nucleotides 1-399 of SEQ ID NO:6 (nucleotides 46-444 of NCBI GenBank Acc. No. M99039) correspond to said first nucleotide sequence encoding the 133-amino acid Qβ CP, Nucleotides 400 to 402 of SEQ ID NO:6 correspond to the strong TAA stop codon and nucleotides 403 to 405 of SEQ ID NO:6 to the leaky TGA stop codon, which is followed by said second nucleotide sequence (Qβ A1). Surprisingly, it was found that the presence of the nucleotide sequence relating to A1 in the expression construct results in higher RNA stability and, thus, in improved yield of Qβ CP and VLP as compared to a construct wherein the A1 sequence is deleted.

The expression of a recombinant protein can significantly reduce the growth rate of the bacterial host due to toxic effects of the accumulating protein and due to the metabolic burden caused by the protein synthesis. In particular cell lysis and low plasmid retention may occur. Inducible promoters provide for the possibility to separate the growth phase from the production phase of a fermentation process. Inducible promoters are repressed by a repressor molecule during the growth phase of the bacterial host and are induced by exposing the bacterial host to inductive conditions during the production phase. Inducible promoters therefore allow the bacterial host to grow fast, preferably exponentially during the growth phase and to reach high cell densities. Thus, inducible promoters provide for high yield of the expression product at the end of the production phase. Therefore, the usage of inducible promoters for the expression of recombinant protein is preferred.

A well known example for an inducible promoter is the lac promoter which forms part of the lac operon and which can be induced by addition of lactose or the strong synthetic inducer isopropylthio-β-D-galactosid (IPTG) to the growth medium of the bacterial host. Donavan et al. 2000 (Can. J. Microbiol 46:532-541) report on an improved process for the expression of a monoclonal antibody fragment under the control of the lac promoter. Further examples of inducible promoters are provided in table 1 of Makrides 1996 (Microbiological Reviews, p. 512-538).

A typical drawback of expression systems based on inducible promoters is the “leakiness” of the promoter, meaning that the promoter is only insufficiently repressed and causes a certain expression rate of the recombinant protein during the growth phase. This typically leads to a reduced cell density or to plasmid instability and, as a consequence, to reduced yield of the recombinant protein Makrides 1996 (Microbiological Reviews, p. 512-538). An example of a promoter which is prone to insufficient repression is the VHb promoter which is repressed under high oxygen conditions and induced upon oxygen depletion.

For the purpose of the invention promoters are preferred which are stringently repressed. In one embodiment the promoter is repressed by the repressor lacI. Examples of such promoters are disclosed in Makrides 1996 (Microbiol. Rev. 60:512-538), Goldstein & Doi 1995 (Biotechnology Annual Review 1:105-128), Hannig & Makrides 1998 (TIBTECH 16:54-60) and Stevens 2000 (Structures 8, R177-R185). In a preferred embodiment the promoter is inducible by lactose, more preferably it is selected from the group consisting of lac, lacUV5, tac, trc, P_{syn}lpp^{a}, lpp-lac, T7-lac, T3-lac, and T5-lac. Especially preferred for the purpose of the invention is the tac promoter (SEQ ID NO:2) or a mutation or variant thereof. Within the scope of the invention are mutants or truncated or deleted variants of the tac promoter having a sequence homology with SEQ ID NO:2 which is at least 50%, 60%, 70%, 80, 90, or 95%, preferably 98 to 100%, most preferably 99%. Wherein the promoter strength of such mutated truncated or deleted variant is comparable to that of the promoter of SEQ ID NO:2. The skilled person will be able to determine the promoter strength of a given sequence by comparative expression studies using standard methods. In a specific embodiment of the invention the promoter driving the expression of the recombinant capsid protein comprises or alternatively consists of SEQ ID NO:2. The tac promoter is a fusion product of the −10 region of the lacUV5 promoter and the −35 region of the trp promoter and combines the high transcription efficiency of trp with the regulatory elements of the lac promoter (de Boer et al. 1983, PNAS 80:21-25; Amann et al. 1983 Gene 25:167-178). It provides for sufficiently high expression rates and high protein yield while avoiding the formation of insoluble or incorrectly folded recombinant protein which may occur with stronger promoters, such as the T7 promoter. The trc and the tic promoter are mutated versions of the tac promoter (Brosius et al. 1985, The Journal of Biological Chemistry 260(6):3539-3541). In a further preferred embodiment the promoter is selected from the group consisting of tic, trc and tac.

For the construction of an expression construct for the purpose of the invention the promoter is operably linked to said first nucleotide sequence encoding the recombinant capsid protein via a ribosome binding site (Shine-Dalgarno sequence, SD), typically comprising an ATG start codon at its 3′ end. Suitable Shine-Dalgarno sequences for the purpose of the invention are well known in the art (Dalbøge et al. 1988, DNA 7(6):399-405; Ringquist et al. 1992, Mol. Micr. 6:1219-1229). In one embodiment of the invention the expression construct comprises the SD sequence of Dalbøge et al. 1988 (DNA 7(6):399-405) which is depicted in SEQ ID NO:4. In another, preferred, embodiment the expression construct comprises a Shine-Dalgarno sequence of Ringquist et al. 1992 (Mol. Micr. 6:1219-1229, SEQ ID NO:3, nSD). Surprisingly, it was found that SEQ ID NO:3 is particularly suited for the purpose of the invention because it results in improved expression levels and improved yield of recombinant capsid protein. SEQ ID NO:3 is especially suited to enhance the expression of AP205 capsid protein. In a preferred embodiment of the invention the expression construct comprises a Shine-Dalgarno sequence selected form the group consisting of SEQ ID NO:3 and SEQ ID NO:4, preferably said Shine-Dalgarno sequence is SEQ ID NO:3.

Transcriptional terminators are functional elements of expression constructs. The skilled person will be able to choose a suitable terminator sequence form a wide range of sources. In a preferred embodiment of the invention said expression construct comprises a terminator sequence, wherein preferably said terminator sequence is operably linked to said first nucleotide sequence, wherein further preferably said terminator sequence is the rRNB terminator sequence, most preferably SEQ ID NO:28.

For the purpose of plasmid selection the skilled person will typically use an antibiotic resistance marker gene. Examples of antibiotic resistance genes which are widely used in the art and which are suitable for the purpose of the invention are resistance genes against the antibiotics ampicillin, tetracyclin and kanamycin. The use of kanamycin as a selective agent in the frame of a process for the production of a VLP is generally preferred because of the lower allergenic potential of kanamycin as compared to alternative antibiotics and because of the lower safety concerns resulting thereof for the use of the VLP as a vaccine. Furthermore, kanamycin provides better plasmid retention as compared to alternative antibiotics such as ampicillin. The kanamycin 3′-phosphotransferase gene (SEQ ID NO:29) which is derived from the transposon Tn903 is therefore a particularly useful selectable marker gene.

The addition of antibiotics to the medium is generally undesirable in a commercial production process for cost and safety reasons. In the context of the invention antibiotics, preferably kanamycin, are typically and preferably used for the selection of the expression strain. Media used in the production process are essentially free of antibiotics, in particular kanamycin. However, addition of an antibiotic to precultures used to produce the inoculum for the production culture can improve plasmid retention throughout the process (Example 10).

The skilled person will create expression plasmids comprising expression constructs which are useful for the production of VLPs of bacteriophages by combining the genetic elements described above applying standard methods of molecular biology. Particularly useful expression plasmids for the purpose of the invention are pTac-nSDQb-mut (SEQ ID NO:1) for the production of Qβ VLP and pTac-nSDAP205 (SEQ ID NO:30) for the production of AP205 VLP. The construction of these specific expression plasmids is described in detail in the Examples section.

The expression plasmids are transformed to a bacterial expression host by any method known in the art, preferably by electroporation. Individual clones of the host comprising the expression plasmid are selected for maximal expression of the recombinant capsid protein by SDS-PAGE after cell lysis. Selected clones of the expression host comprising the expression plasmid can be stored as frozen glycerol cultures.

Said bacterial host can be chosen from any bacterial strain capable of replicating and maintaining said expression plasmid during cell division. Preferred bacterial hosts are Escherichia coli strains having the specific features described in the following sections.

The repression of the promoter is improved by overexpression of the repressor by the bacterial host. In one embodiment said cultivating of said bacterial host is performed in batch culture and under conditions under which said promoter is repressed by lacI. In a preferred embodiment the gene causing overexpression of said lacI in said bacterial host is located on a plasmid, preferably on said expression plasmid. Alternatively, said gene is located on a separate plasmid contained in said bacterial host, wherein said separate plasmid preferably is a high copy number plasmid. Alternatively, and most preferably said gene is located on the chromosome of said bacterial host.

One example of a gene causing overexpression of lacI is lacI^{q} (Menzella et al. 2003, Biotechnology and Bioengineering 82(7)809-817) which is a single CG to TA change at −35 of the promoter region of lacI which causes a 10 fold increase in LacI expression. A further example is lacIQ1 (Glascock & Weickert 1998, Gene 223(1-2):221-231). Improved repression of the promoter during the growth phase results in improved plasmid retention and higher cell density and, ultimately, in improved protein yield. For example, bacterial strains comprising the lacI^{q} gene overexpress the lacI repressor molecule and therefore prevent formation of the recombinant protein during the growth phase more efficiently than strains comprising the wildtype gene. In a preferred embodiment the gene causing overexpression of said lacI is lacIQ1 or lacI^{q}, preferably lacI^{q}. In a specifically preferred embodiment said bacterial host comprises the lacI^{q} gene on its chromosome.

In one embodiment said inducing of said promoter is performed with an inducer, wherein said inducer is preferably selected from IPTG and lactose, most preferably said inducer is lactose. Upon exposure of the bacterial host to an inducer, the repressor is inactivated and the promoter becomes active. Addition of the strong inducer IPTG to the culture medium results in an immediate increase of the expression rate of the recombinant protein to a high level because IPTG directly enters the cells by diffusion and binds and inactivates the active repressor lacI. Inactivated lacI repressor molecules dissociates from the operator and allow high level transcription from the promoter. IPTG is not metabolized by the cell and the transcription continues with high rates until other metabolic parameters become limiting.

As mentioned before, high expression rates may lead to the formation of insoluble recombinant protein which is not capable of forming a VLP by self-assembly. Induction of protein expression with high concentrations of IPTG is particularly prone to the formation of insoluble protein. Therefore, induction of the promoter is preferably achieved by the addition of IPTG in concentrations which are below the concentration which causes the expression to occur at its maximum rate (Kopetzki et al. 1989, Mol Gen Genet 216:149-155).

In a preferred embodiment said inducing of said promoter is performed with IPTG, wherein the concentration of said IPTG in said medium is about 0.001 to 5 mM, preferably 0.001 to 1 mM, more preferably 0.005 to 1 mM, still more preferably 0.005 to 0.5 mM. In a specifically preferred embodiment the concentration of said IPTG is about 0.01 mM, most preferrably 0.01 mM.

Alternatively, induction of the promoter is achieved by the addition of lactose. Induction of recombinant protein expression with lactose requires that the bacterial host is capable of taking up lactose from the medium, e.g. by Lac permease and that it comprises β-galactosidase activity. The Lac permease dependent uptake of lactose into the cells follows a slower kinetic than the uptake of IPTG by diffusion. Furthermore, lactose does not directly interact with the lac operon but is converted by β-galactosidase to allolactose (1-6-O-β-galactopyranosyl-D-glucose) which is the actual inducer of the promoter. Induction of recombinant protein expression by the addition of lactose is advantageous because it avoids the immediate increase of the expression rate to a maximum level upon addition of the inducer and, thus, it reduces the risk of the formation of insoluble protein.

Allolactose is metabolised by the bacterial host during the production phase and contributes carbon and metabolic energy to the bacterial metabolism. This may further contribute to improved protein yield as compared to induction with IPTG. Furthermore, induction by lactose allows to a certain extend the control of the expression rate of the recombinant protein during the production phase via the lactose concentration in the medium. Induction by lactose is further preferred in a pharmaceutical production process because IPTG is expensive and is believed to be toxic. Its removal needs to be demonstrated at the end of a the production process.

In a preferred embodiment said inducing of said promoter is performed by the addition of lactose to said batch culture, wherein preferably said bacterial host is capable of taking up lactose from the medium and wherein further preferably said bacterial host comprises β-galactosidase activity. Such bacteria strains can, for example, be obtained from strain collections such as ATTC (http://www.atcc.org). In a preferred embodiment, said bacterial host is an E. coli strain, preferably an E. coli strain selected from the group consisting of RB791, DH20, Y1088, W3110 and MG1655. Most preferably, said bacterial host is E. coli RB791. In a still more preferred embodiment said promoter is the tac promoter or mutant or variant thereof and said bacterial host is an E. coli strain which further comprises a gene causing overexpression of a repressor of the tac promoter, wherein said gene preferably is lacI^{q}. The pH of the culture medium of the bacterial host can be controlled during the fermentation process and regulated by the addition of acidic or alkaline solutions using methods which are well known in the art. In one embodiment, said cultivating of said bacterial host and said feeding of said batch culture is performed under conditions, wherein the pH of the medium is controlled. In a preferred embodiment said pH is between 5.5 and 8.0, more preferably between 6.5 and 7.5, even more preferably between 6.7 and 7.0 and most preferably said pH of said medium is 6.8. Said pH of said medium may be kept constant during the process or it may follow a certain profile during the different phases of the process within the pH ranges specified above. In a preferred embodiment said pH is kept constant at a value of 6.7 to 7.0, preferably said pH it is kept constant at 6.8.

The process of the invention comprises a growth phase, wherein said growth phase comprises a batch and a feed phase, wherein said growth phase and simultaneously said batch phase are initiated by said cultivating said bacterial host and wherein said feed phase is initiated by said feeding of said batch culture with said major carbon source.

The oxidative capacity of bacteria cells is limited and high concentrations of the substrate may cause the formation of reduced products like acetate, which may lead to undesired acidification of the medium and to reduced growth of the bacteria. Therefore, the bacterial host is grown in a fed-batch culture on a minimum medium with a limited quantity of substrate. In one embodiment said cultivating of said bacterial host is performed in a medium comprising said major carbon source, wherein said medium preferably is a minimal medium, preferably a chemically defined minimal medium. Most preferably said medium is R27 medium as described in Example 5.

At the end of the batch phase, when the substrate contained in the medium is almost exhausted, medium containing the major carbon source (feed medium) is fed to said batch culture at the same rate as the desired growth rate of the bacterial host, i.e. the growth rate of the bacterial host is limited by the feed rate of the substrate. It is understood by the skilled person that the decisive parameter is the actual mass flow of the substrate, preferably the major carbon source, and other nutrients required to maintain growth. Since in practice a constant composition of the feed medium can be assumed, the flow rate refers to the volume flow of the medium. The same consideration applies to the co-feed medium (see below).

Therefore, in one embodiment said feeding of said batch culture with said major carbon source is performed with a flow rate, wherein said flow rate is limiting the growth rate of said bacterial host.

During the feed phase the growth rate can be freely selected in a wide range nearly up to the maximum growth rate (μₘₐₓ) if no inhibition occurs. The actual value of μₘₐₓ is highly dependent on the bacterial strain, the expression construct and the growth conditions. The skilled person will understand that the determination of μₘₐₓ is performed under conditions under which the promoter is repressed.

For a given experimental set up, μ can be determined from the growth curve of the culture by plotting biomass concentration (x) as determined by OD₆₀₀ or cell wet weight (CWW) against the cultivation time and determining the exponential growth coefficient μ based on the equation x=x₀e^{μt} The actual value of μₘₐₓ is determined as the growth rate μ of an exponentially growing batch culture in the beginning of the batch phase when no substrate limitation occurs, i.e. without supply of additional medium by feeding. The growth rate μ can be determined by computing the ratio of the difference between natural logarithm of the total biomass X₂ measured at time t₂ and natural logarithm of the total biomass X₁ measured at time t₁ to the time difference (t₂−t₁): μ=(1nX₂−lnX₁)/(t₂−t₁).

Fed-batch culture allows the maintenance of a constant growth rate (p). In a preferred embodiment the substrate, preferably the major carbon source, is fed during the feed phase according to the exponential increase of the biomass (x). If during the feed phase the substrate is supplied at the same rate it is consumed, the culture is in a quasi steady state, analogous to the cultivation in a continuous culture. Because biomass formation and substrate consumption are correlated over the substrate-referred yield coefficient Y_{x/s} (biomass [g]/substrate [g]), the substrate quantity (s) per time unit (t) to be supplied is calculated according to the formula ds/dt=μ/Y_{x/s}x_{0 tot}e^{μt}, wherein x_{0 tot} is the total biomass at feed start.

Therefore, in a preferred embodiment said feeding of said batch culture with said major carbon source is performed with a flow rate, wherein said flow rate increases with an exponential coefficient μ, and wherein preferably said exponential coefficient μ is below μmax. Thus, the growth rate of said bacterial host during the feed phase is set to a value which is below μₘₐₓ. In a preferred embodiment said exponential coefficient μ is about 30% to 70%, most preferably about 50% of μₘₐₓ. In a specific embodiment of the invention μ is set to an absolute value of 0.15 to 0.45 h⁻¹, more preferably 0.25 to 0.35 h⁻¹, most preferably μ is 0.3 h⁻¹, provided that the set up of the process is such, that these values are below μₘₐₓ.

Bacteria are able to utilise a wide range of different substrates. For the purpose of the invention, preferred major carbon sources are glucose and glycerol, preferably glycerol. Although the maximum specific growth rate (μₘₐₓ) of the expression host which can be achieved may be higher with glucose than with glycerol, glycerol causes less acetate formation and provides higher biomass yield per substrate (Y_{x/s}) and, ultimately, higher yield of the recombinant protein. Furthermore, the handling of the liquid substrate glycerol is easier than that of solid carbon sources like glucose which need to be dissolved in a separate process step.

As mentioned before, plasmid retention, i.e. the maintenance of the expression plasmid in the bacterial host during the fermentation process, is essential for optimal yield of the recombinant protein. Plasmid retention can be assessed by spreading bacteria cells on a solid medium to form single colonies and testing individual colonies for their antibiotic resistance. For example, a plasmid retention of 100% means that 100 out of 100 tested colonies comprise the specific antibiotic resistance which conferred by the expression plasmid. For the purpose of the invention plasmid retention at the end of the fermentation process is more than 80%, preferably more than 90%, more preferably more than 95%, even more preferably more than 97% and most preferably 100%.

The optimal growth temperature of a bacterial strain is the temperature at which it reaches its highest maximal growth rate (μₘₐₓ). Under otherwise not limiting conditions for most E. coli strains this temperature is about 37° C. However, growth of the bacterial strain comprising the expression construct at the optimal growth temperature and in the absence of a selective antibiotic may favour the loss of the expression plasmid, whereas plasmid retention is generally improved when the expression strain is grown at lower temperature. Although the maximum growth rate of the expression strain is lower when the strain is grown at temperatures below its optimal growth temperature as compared to growth at the optimal growth temperature, the yield of recombinant protein may be equal or even better at the lower temperature due to improved plasmid retention.

In one embodiment of the invention, said cultivating of said bacterial host and/or said feeding of said batch culture with said major carbon source and/or said inducing said promoter with an inducer is therefore performed at a temperature below the optimal growth temperature of said bacterial host. In a preferred embodiment said temperature is between 2° and 37° C., preferably between 23 and 35° C., more preferably between 25 and 33° C., even more preferably between 27 and 32° C., still more preferably between 28 and 31° C. Still more preferably said temperature is about 30° C., most preferably said temperature is 30° C.

The process of the invention comprises a production phase, wherein said production phase is initiated by said inducing said promoter with an inducer. The time point for the initiation of said production phase can be determined based on cultivation time and/or growth parameters.

The growth of the bacterial host during the fermentation process can be assessed by determining the optical density at 600 nm (OD₆₀₀), the cell wet weight (CWW [g/l]) and the cell dry weight (CDW [g/l]). These parameters can be used to define the optimal time point for the start of the production phase by addition of the inducer, preferably lactose, to the medium. It is apparent for the skilled person, that on one hand higher CWW at the beginning of the production phase can be achieved by an extended feed phase and may lead to improved yield of the recombinant protein but that on the other hand over-aged cultures may show insufficient protein expression. The optimal time point for the beginning of the production phase, which is initiated by said inducing of said promoter with an inducer, therefore needs to be determined for the specific production conditions. For example, for expression of Qβ CP in E. coli RB791 in a total volume of 2 l, induction is started after ca. 14 h, when OD₆₀₀ has reached about 40 to 60. Surprisingly, similar parameters were found for the same process in a 50 l scale, where induction start is also after ca. 14 h when OD₆₀₀ has reached about 50.

Therefore, in one embodiment of the invention, said inducing of said promoter with said inducer is performed 10 h to 16 h after the beginning of said growth phase, preferably after 12 h to 15 h, more preferably after 13 h to 15 h, most preferably after about 14 h, wherein preferably said inducing of said promoter with said inducer is performed when the OD₆₀₀ has reached about 40 to 60, preferably about 50.

In a further embodiment, said inducing of said promoter with said inducer is performed after an extended feed phase, wherein preferably said inducing of said promoter with said inducer is performed 14 h to 20 h after the beginning of said cultivating of said bacterial host in a medium, preferably after 15 h to 18 h, more preferably after 16 h to 17 h, most preferably after about 16.5 h, wherein preferably said inducing of said promoter with said inducer is performed when the OD₆₀₀ has reached about 80 to 90, preferably about 85.

In one embodiment of the invention said inducing of said promoter with said inducer is performed when the OD₆₀₀ reached a value of 25 to 60, preferably 25 to 55, more preferably 30 to 50, most preferably 30 to 40. In a specifically preferred embodiment said inducing of said promoter with said inducer is performed when OD₆₀₀ is 35.

In another embodiment of the invention said inducing of said promoter with said inducer is performed after an extended feed phase, when the OD₆₀₀ reached a value of 60 to 120, preferably 70 to 110, more preferably 80 to 100, most preferably 80 to 90. In a specifically preferred embodiment the induction is started after and an extended feed phase when OD₆₀₀ is about 85, preferably 85.

Induction with IPTG: In one embodiment of the invention said inducing of said promoter with an inducer is achieved by the addition of IPTG, wherein preferably said feeding of the culture with the major carbon source is continued. Since IPTG is not metabolized by the bacterial host, induction can be achieve by a single addition of IPTG to the desired concentration. Alternatively, induction can be achieved by a continuous flow of IPTG to the culture. In a preferred embodiment induction is performed by addition of IPTG in a single addition or a continuous flow, wherein said feeding of the batch culture with the major carbon source is continued with a constant or an increasing flow rate of said major carbon source exponentially increasing flow rate of the major carbon source.

Induction with lactose: As described above, the induction of protein expression can alternatively be achieved by the addition of lactose to the culture medium. In one embodiment of the invention, at the beginning of the production phase the exponential feed of the substrate is interrupted and the culture is supplied with a constant flow of induction medium containing 100 to 300 g/l, preferably 100 g/l lactose as the sole carbon source (lactose feed medium). Preferably, the constant flow rate of lactose equals approximately the flow rate of the substrate at the end of the feed phase.

In a preferred embodiment of the invention said inducing of said promoter with an inducer is achieved by the addition of lactose, wherein preferably said lactose is fed to said batch culture in a continuous flow during and wherein preferably said feeding of said batch culture with said major carbon source is not continued.

Upon addition of lactose to the culture, the β-galactosidase activity increases, lactose is converted to allolactose which induces the tac promoter and the expression of the recombinant capsid is initiated. In parallel, allolactose is further metabolised and contributes to the energy supply for the bacterial host. The equilibrium of the feeding rate of the induction medium and the lactose consumption by the cells thus determines the expression rate. The enzymatic reactions involved in this cascade allow to control the process in such a way that the formation of inclusion bodies is minimised. The progress of induction process can be monitored by determining the β-galactosidase activity in the culture, e.g. by a β-Gal Assay Kit (Invitrogen, K1455-01).

In a more preferred embodiment of the invention said inducing of said promoter with an inducer is achieved by the addition of lactose, wherein preferably said lactose is fed to said batch culture in a continuous flow during and wherein preferably said feeding of said batch culture with said major carbon source is continued.

Discontinuous addition of inducer: Said inducer can be added to the culture discontinuously by a single addition at the beginning of the production phase or by a few subsequent additions during the production phase. Discontinuous addition of the inducer, especially by a single addition is particularly suited when the inducer is IPTG since IPTG is not metabolized by the bacterial host. Therefore, typically and preferably no replacement of metabolised IPTG is necessary during the production phase. In one embodiment said inducing of said promoter with an inducer is performed by the addition of said inducer, preferably IPTG or lactose, most preferably IPTG, to said medium, wherein said inducer is added to about its final concentration at once by a single addition at the beginning of the production phase, wherein preferably said feeding of said batch culture with said major carbon source is continued. In a preferred embodiment said inducing of said promoter with an inducer is performed by the addition of IPTG to said medium, wherein said IPTG is added to about its final concentration at once by a single addition, wherein preferably said feeding of said batch culture with said major carbon source is continued. Alternatively, said inducing of said promoter with an inducer is performed by the addition of said inducer, preferably IPTG or lactose, most preferably lactose, to said medium, wherein said addition is performed in several steps, preferably in 1 to 5, more preferably in 2 to 4, most preferably in 3 steps during the production phase, wherein preferably said feeding of said batch culture with said major carbon source is continued.

Continuous addition (feeding) of inducer: Preferably, said inducer is added to the medium in a continuous flow, preferably throughout the production phase. The continuous addition of the inducer is particularly suited for lactose, since lactose is metabolised by the bacterial host and therefore a continuous addition of lactose during the production phase allows to maintain a lactose concentration in the medium which allows for efficient induction of the promoter. In a preferred embodiment, said inducing of said promoter with an inducer is performed by feeding said batch culture with said inducer, wherein preferably said inducer is IPTG or lactose, most preferably lactose, and wherein said feeding is performed in a continuous flow, wherein further preferably said feeding is performed throughout the production phase.

Co-feeding of inducer and major carbon source: The expression of the recombinant protein is an energy demanding process. To prevent yield loss which might be caused by the excessive consumption of the inducer by the bacterial host and low expression rates resulting thereof, the culture can be additionally supplemented with substrate, preferably the major carbon source, during the production phase, wherein the flow rate of inducer and/or the major carbon source is constant or increasing, preferably constant. When during the production phase the culture is supplemented with substrate at an increasing flow rate, the flow rate is preferably increasing with an exponential rate.

Co-feeding with constant flow rate: In a preferred embodiment said inducing of said promoter with an inducer is performed by co-feeding said batch culture with said inducer and said major carbon source, wherein said inducer is preferably IPTG or lactose, most preferably lactose, and wherein said major carbon source is glucose or glycerol, preferably glycerol, wherein said inducer, preferably lactose and said major carbon source, preferably glycerol are co-fed to said batch culture at a flow rate, wherein said flow rate is preferably about constant. In a further preferred embodiment said flow rate is chosen to allow feeding of said major carbon source to said batch culture at about the same rate as at the end of the growth phase. In a still further preferred embodiment said inducer, preferably lactose, and said major carbon source, preferably glycerol, are contained in the same medium (co-feed medium). In a further preferred embodiment said co-feed medium is fed to said batch culture with a flow rate, wherein said flow rate is preferably about constant, and wherein further preferably said flow rate is chosen to allow feeding of said major carbon source to said batch culture at about the same rate as at the end of the growth phase. In a very preferred embodiment said inducer is lactose and said major carbon source is glycerol, wherein said lactose and said glycerol are co-fed to said batch culture in a ratio of about 2:1 to 1:4 (w/w).

In a further preferred embodiment of the invention lactose and said major carbon source, preferably glycerol, are co-fed to said batch culture in a ration of 0:1 to 1:0 (w/w), preferably about 2:1 to about 1:4 (w/w), more preferably about 1:1 to 1:3 (w/w), most preferably the ratio is about 1:3 (w/w). In a preferred embodiment the ratio of lactose and the major carbon source, preferably glycerol, is 1:1 (w/w). In another preferred embodiment the ratio of lactose and the major carbon source, preferably glycerol, is 1:3 (w/w). In a more preferred embodiment said co-feed medium comprises ca. 200 g/l lactose and ca. 200 g/l glycerol. In a still more preferred embodiment the co-feed medium comprises ca. 100 g/l lactose and ca. 300 g/l glycerol.

Co-feeding with increasing flow rate: Alternatively, said inducing of said promoter with an inducer is performed by co-feeding said batch culture with said inducer and said major carbon source, wherein said inducer is preferably IPTG or lactose, most preferably lactose, and wherein said major carbon source is glucose or glycerol, preferably glycerol, wherein said inducer, preferably lactose and said major carbon source, preferably glycerol are co-fed to said batch culture at a flow rate, wherein said flow rate is increasing, wherein said flow rate may increase with a linear or with an exponential characteristic, wherein preferably the initial flow rate is chosen to to allow feeding of said major carbon source to said batch culture at about the same rate as at the end of the growth phase.

Further alternatively said inducing of said promoter with an inducer is performed by co-feeding said batch culture with said inducer and said major carbon source, wherein said inducer is preferably IPTG or lactose, most preferably lactose, and wherein said major carbon source is glucose or glycerol, preferably glycerol, wherein said inducer, preferably lactose is fed to said batch culture at a first flow rate, and wherein said major carbon source, preferably glycerol is fed to said batch culture at a second flow rate, wherein said first flow rate is constant or increasing, preferably constant, and wherein said second flow rate is constant or increasing, preferably increasing, wherein preferably the initial value of said second flow rate is chosen to to allow feeding of said major carbon source to said batch culture at about the same rate as at the end of the growth phase. In a very preferred embodiment said inducer is lactose and said major carbon source is glycerol, wherein said lactose and said glycerol are co-fed to said batch culture in a ratio of about 2:1 to 1:4 (w/w).

The growth of the bacterial host as determined by CDW, CWW or OD₆₀₀ continues during the production phase at a growth rate which is lower than that during the growth phase and which is decreasing with the process time. In a further embodiment of the invention, said inducing said promoter with an inducer is performed by co-feeding said inducer, preferably lactose and said major carbon source, preferably glycerol, to said batch culture with an increasing flow rate, preferably with a flow rate wherein the incremental increase of the flow rate is adapted to the actual growth rate of the culture. In a further preferred embodiment said inducer, preferably lactose, and said major carbon source, preferably glycerol, are contained in the same medium (co-feed medium), wherein preferably the ratio between lactose and glycerol in said medium (co-feed medium) ranges from about 0:1 to 1:0 (w/w), preferably about 2:1 to about 1:4 (w/w), more preferably about 1:1 to 1:3 (w/w), most preferably the ratio is about 1:3 (w/w). In a preferred embodiment the ratio of lactose and the major carbon source, preferably glycerol, is 1:1 (w/w). In another preferred embodiment the ratio of lactose and the major carbon source, preferably glycerol, is 1:3 (w/w). In a more preferred embodiment said medium (co-feed medium) comprises ca. 200 g/l lactose and ca. 200 g/l glycerol. In a still more preferred embodiment the induction medium comprises ca. 100 g/l lactose and ca. 300 g/l glycerol.

In one embodiment of the invention said inducing of said promoter with an inducer is performed by co-feeding said inducer, preferably lactose and said major carbon source, preferably glycerol to said batch culture, wherein said inducer, preferably lactose and said major carbon source, preferably glycerol are contained in separate media which are separately fed to said culture.

At the end of the production phase the cells are harvested by centrifugation. Typically, cells are harvested about 5 h after induction start, when a final OD₆₀₀ of 90 to 130 is reached. Further extension of the production phase leads to higher OD₆₀₀ and CWW values and therefore to further improved yield of the expression construct.

Harvested cells may be suspended in a storage buffer and stored at −80° C. for further processing.

The total protein content of the cells is determined after cell lysis by SDS PAGE or LDS PAGE and comparison with a protein standard. The content of soluble protein is determined by HPLC. The identity of the expressed capsid protein is determined by western blotting. The concentration of assembled VLPs can be analysed by size exclusion chromatography (Example 18). VLP can preparatively be purified from lysed cells by chromatographic methods.

Scale-up of the process of the invention to large volumes is possible with only minor adaptations. The invention encompasses culture volumes in the range of 100 ml up to 6000 l. Preferred culture volumes are 40 to 100 l, most preferably about 50 l. It is apparent for the skilled person that larger culture volumes in particular require larger volumes of the preculture which is used for inoculation. For example, a preculture may be performed in two ore more steps with increasing preculture volume. To ensure plasmid retention in large culture volumes, the precultures which are used as inoculum may contain an antibiotic to maintain selection pressure. The skilled person is aware that plasmid retention can further be improved by reducing the number of generations which is necessary to reach the desired final cell density. Therefore, it is advantageous to inoculate the precultures and the batch cultures with high cell densities. In a preferred embodiment the initial OD₆₀₀ of the preculture is 0.1 to 0.4, preferably about 0.3.

In one embodiment, prior to said cultivation step, said process further comprises the step of introducing said bacterial host into a medium, wherein said introducing is performed with an inoculum, wherein said inoculum is produced in a preculture process comprising the step of growing said bacterial host in a medium comprising an antibiotic, preferably kanamycin. More preferably, said preculture process comprises the steps of growing said bacterial host in a first medium comprising an antibiotic, preferably kanamycin, and diluting said first medium comprising the bacterial host with a second medium to an OD₆₀₀ of 0.1 to 0.4, preferably about 0.3, wherein said second medium is essentially free of an antibiotic, and further cultivating said bacterial host.

Furthermore, it is apparent for the skilled person, that the fermentation process of the invention is an aerobic process which requires adequate oxygen supply of the bacteria in the culture. The oxygen demand of the bacterial host is, inter alia, increasing with increasing cell density and increasing growth rate. Depending on the total volume and the oxygen demand of the bacterial host, oxygen can, for example, be supplied by stirring and/or by aeration with air. Alternatively, oxygen can also be supplied by aeration with pure oxygen or a mixture of pure oxygen with any other gas, preferably air, wherein pure oxygen refers to the technically pure gas as commonly available for technical purposes. A further possibility of supplying oxygen to the bacterial host is increasing the oxygen partial pressure in the medium by increasing the pressure in the fermenter.

In a preferred embodiment of the invention, said cultivating said bacterial host and/or said feeding of said batch culture and/or said inducing of said promoter with an inducer is performed under conditions, wherein said bacterial host is supplied with oxygen, preferably by aeration with air, most preferably by aeration with air in a constant flow, wherein preferably said oxygen is supplied throughout the entire process, most preferably throughout the lag-, growth- and production phase, and wherein further preferably the partial pressure of oxygen is monitored in the culture medium and wherein the bacterial host is alternatively or additionally supplied with oxygen by aeration with pure oxygen, preferably when the partial pressure of oxygen in the medium (pO₂) is below a certain threshold. In a specifically preferred embodiment said threshold of pO₂ is in the range of 0% to 60%, preferably 10% to 50%, more preferably 20% to 45% most preferably said threshold is about 40%.

Oxygen supply, preferably by aeration with air and/or pure oxygen to maintain the preferred pO₂ as described above, is routinely applied in the process of the invention, preferably for culture volumes of 21 and more. Aeration with oxygen in the described manner is especially preferred in the scaled-up process, most preferably at 40 to 1001 and above.

Therefore, one embodiment of the invention is a process for expression of a recombinant capsid protein of a bacteriophage or a mutant or fragment thereof being capable of forming a VLP by self-assembly, said process comprising the steps of: a) introducing an expression plasmid into a bacterial host, wherein said expression plasmid comprises an expression construct, wherein said expression construct comprises (i) a first nucleotide sequence encoding said recombinant capsid protein, or mutant or fragment thereof, and (ii) a promoter being inducible by lactose; b.) cultivating said bacterial host in a medium comprising a major carbon source; wherein said cultivating is performed in batch culture and under conditions under which said promoter is repressed by lacI, wherein said lacI is overexpressed by said bacterial host; c.) feeding said batch culture with said major carbon source; and d.) inducing said promoter with an inducer, wherein said feeding of said batch culture with said major carbon source is continued; wherein throughout steps b.) to d.) of said process oxygen is supplied to said bacterial host by a pO₂ in said medium of at least about 10% to 50%, preferably about 40%, and wherein further preferably said oxygen is supplied by aeration with air, pure oxygen, or a mixture of both, preferably by a mixture of air and pure oxygen.

### EXAMPLES

### Example 1

Cloning Strategy for the Expression Plasmid pTac-nSD-Qb-mut (SEQ ID NO:1) The coat protein-encoding gene (C) of E coli RNA bacteriophage Qβ is amplified from plasmid pSDQb-mut (SEQ ID NO:33). The plasmid contains the sequence of gene C coding for the 133-aa Qβ coat protein (CP) and the 329-aa read through protein (A1). To prevent read-through, nucleotides 445-450 according to NCBI GenBank Acc. No. M99030 TGAACA (SEQ ID NO:31) are replaced by the sequence TAATGA (SEQ ID NO:32).

The coat protein-encoding gene C from plasmid pSDQb-mut is amplified by PCR. Oligonucleotide Qb-FOR3/2 (SEQ ID NO:34) with an internal EcoRI site and a synthetic Shine-Dalgarno (SD, SEQ ID No: 4) sequence anneals to the 5′ end of the Qβ CP gene. Oligonucleotide Qblang-REV2/2 (SEQ ID NO:35) contains an internal HindIII site and primes to the 3′ end of the noncoding region of gene C. The 1054 bp amplified PCR fragment includes nucleotides 46-1062 of NCBI GenBank Acc. No. M99039 (except the nucleotide changes described above) and the synthetic SD sequence. The PCR fragment is digested with the restriction enzymes HindIII/EcoRI and the resulting 1036 bp fragment is inserted into the HindIII/EcoRI restriction sites of a modified pKK223-3 vector (Pharmacia, NCBI GenBank Acc. No.: M77749, SEQ ID NO:27). In this modified pKK223-3 vector the ampicillin resistance gene is replaced with the kanamycin resistance gene of vector pUC4K (Pharmacia, NCBI GenBank Acc. No.: X₀₆₄₀₄, SEQ ID NO:37).

Vector pTac-nSDQb-mut (SEQ ID NO:33) differs from vector pTacQb-mut in the Shine-Dalgarno sequence. This Shine-Dalgarno sequence (nSD, SEQ ID NO:3) is introduced by amplifying the Qβ coat protein-encoding gene C via PCR from plasmid pTacQb-mut. Oligonucleotide nSDQb-mutEcoRIfor (SEQ ID NO:36) with an internal EcoRI site and the corresponding synthetic Shine-Dalgarno (nSD) sequence anneals to the 5′ end of the Qβ CP gene. Oligonucleotide Qblang-REV2/2 (SEQ ID NO:35) contains an internal HindIII site and primes to the 3′ end of the noncoding region of gene C. The 1054 bp amplified PCR fragment includes nucleotides 46-1062 of NCBI GenBank Acc. No. M99039 (except the nucleotide changes described above) and the synthetic nSD sequence. The PCR fragment is digested with the restriction enzymes HindIII/EcoRI and the resulting 1036 bp fragment is inserted into the HindIII/EcoRI restriction sites of a modified pKK223-3 vector (Pharmacia, NCBI GenBank Acc. No.: M77749, SEQ ID NO:27). In this modified pKK223-3 vector the ampicillin resistance gene is replaced with the kanamycin resistance gene of vector pUC4K (Pharmacia, NCBI GenBank Acc. No.: X₀₆₄₀₄, SEQ ID NO:37).

### Example 2

Cloning Strategy for the Expression Plasmid pTac-nSD-AP205 (SEQ ID NO:30) The coat protein-encoding gene of Acinetobacter bacteriophage AP205 is amplified from plasmid pAP205-58. This plasmid contains the sequence of the coat protein gene (corresponding to nucleotides 1908-2303 of NCBI GenBank Acc. No. AF334111) coding for the 131-amino acid capsid protein of bacteriophage AP205.

The coat protein-encoding gene is amplified by PCR. Oligonucleotide nSDAP238-EcoRIfor (SEQ ID NO:38) with an internal EcoRI site and a synthetic Shine-Dalgarno (nSD) sequence anneals to the 5′ end of the coat protein gene. Oligonucleotide AP238HindIIIrev (SEQ ID NO:39) contains an internal HindIII site and primes to the 3′ end of the coat protein gene. This oligonucleotid introduces a second stop codon behind the naturally occurring stop codon of the coat protein. The 438 bp amplified PCR fragment includes nucleotides 1908-2303 of NCBI GenBank Acc. No. AF334111 and the synthetic nSD sequence. The PCR fragment is digested with the restriction enzymes HindIII/EcoRI and the resulting 420 bp fragment is inserted into the HindIII/EcoRI restriction sites of a modified pKK223-3 vector (Pharmacia, NCBI GenBank Acc. No.: M77749, SEQ ID NO:27). In this modified pKK223-3 vector the ampicillin resistance gene is replaced with the kanamycin resistance gene of vector pUC4K (Pharmacia, NCBI GenBank Acc. No.: X₀₆₄₀₄, SEQ ID NO:37).

### Example 3

Expression of Qβ CP under control of the tac promoter and nSD The E. coli strain RB791 was transformed with plasmids pTac-nSD-Qb-mut (SEQ ID NO:1). The clone was grown in shake flasks. Each flask contained 100 ml of R40 medium (main culture medium, Hypep 7455, glycerol, see Example 5) with kanamycin (25 μg/ml) and was inoculated with over night cultures at a start OD₆₀₀ of 0.3. The shake flasks were incubated for 4 h (OD₆₀₀ between 4 and 5) at 30° C. and an agitation of 220 rpm. The induction was carried out with 0.5% of lactose for 4 h. Protein production was determined by SDS-PAGE. The gel showed a strong protein band which was identified as Qβ CP.

### Example 4

Expression of AP205 CP under control of the tac promoter and SD vs. nSD 9 clones of pTac-nSDAP205 (SEQ ID NO:30) and 6 clones of pTac-SDAP205 were screened in shake flasks. pTac-SDAP205 (SEQ ID NO:40) is identical to pTac-nSDAP205 but comprises the Shine-Dalgarno sequence of SEQ ID NO:4 instead of that of SEQ ID NO:3. Each flask contained 50 ml of R40 medium (main culture medium, Hypep 7455, glycerol, see Example 5) with kanamycin (25 μg/ml) and was inoculated with over night cultures at a start OD₆₀₀ of 0.3 (for pTac-nSDAP205) or 0.4 (pTac-SDAP205). The shake flasks were incubated for 4 h at 30° C. and an agitation of 220 rpm. The induction was carried out with 0.5% of lactose. Protein production was determined by SDS-PAGE. For all tested clones expression of AP205 CP was significantly stronger from pTac-nSDAP205 than from pTac-SDAP205.

### Example 5

Composition of Culture Media Culture media were composed as described in Table 1.

TABLE 1
Composition of Culture media.

| | Concentrations in [g/L] | | | | |
|---|---|---|---|---|---|
| | | Main | | Induction | Main |
| | Main | Medium + | Feed | Medium + | Medium + |
| | Medium + | Hypep + | Medium + | 20% Glycerol + | Bacto YE + |
| | Hypep | Glycerol | 50% Glycerol | 20% Lactose | Glycerol |
| Component | R27 | R40 | R41 | R42 | R43 |
| Na₂HPO₄ 2H₂O | 2.5 | 2.5 | 2.5 | 2.5 | 2.5 |
| KH₂PO₄ | 3 | 3 | 3 | 3 | 3 |
| K₂HPO₄ | 5.2 | 5.2 | 5.2 | 5.2 | 5.2 |
| Citrate | 3.86 | 3.86 | 3.86 | 3.86 | 3.86 |
| (NH₄)2SO₄ | 4 | 4 | 4 | 4 | 4 |
| Vit B1 | 0.01 | 0.01 | 0.02 | 0.02 | 0.01 |
| CaCl₂ 2H₂O | 0.0147 | 0.0147 | 0.0147 | 0.0147 | 0.0147 |
| MgSO₄ 7H₂O | 0.5 | 0.5 | 9 | 9 | 0.5 |
| FeCl₃ 6H₂O | 0.054 | 0.054 | 0.054 | 0.054 | 0.054 |
| CoCl₂ 6H₂O | 0.0005 | 0.0005 | 0.0005 | 0.0005 | 0.0005 |
| MnCl₂ 4H₂O | 0.003 | 0.003 | 0.003 | 0.003 | 0.003 |
| CuCl₂ 2H₂O | 0.0003 | 0.0003 | 0.0003 | 0.0003 | 0.0003 |
| H₃BO₃ | 0.003 | 0.003 | 0.003 | 0.003 | 0.003 |
| Na₂MoO₄ 2H₂O | 0.0005 | 0.0005 | 0.0005 | 0.0005 | 0.0005 |
| Zn(CH₃COO)₂ 2H₂O | 0.0026 | 0.0026 | 0.0026 | 0.0026 | 0.0026 |
| Glucose | 5 | — | — | — | — |
| Glycerol | — | 5 | 500 | 200 | 5 |
| Lactose anhydrous | — | — | — | 200 | — |
| HyPep 7455 | 5 | 5 | — | — | — |
| Bacto Yeast Extract | — | — | — | — | 5 |

### Example 6

### Expression of Qβ CP in a Fed-Batch Process (2 L Scale)

The fermentation process was performed in a bioreactor (Applikon 5 L dished bottom) equipped with 2 disc stirrer (Ø6 cm), baffles (3×16 cm), pH-, pO2-, and temperature control, and fermenter software BioXpert Version 2.22

5 μL cryo culture of RB791 transformed with plasmids pTac-nSD-Qb-mut were inoculated in 100 mL Erlenmeyer flasks containing 50 mL medium R40 (25 μg/mL kanamycin) and cultivated for 14 h at 30° C. and 220 RPM over night. After 14 h an OD₆₀₀ value of 6.0 was reached. For batch fermentation, 2 L of medium (R40) were pumped into the bioreactor. In Table 2 the cultivation parameters are listed.

TABLE 2
Parameter set points for batch phase.

| Parameter | Set point | Unit |
|---|---|---|
| Stirrer speed | 1000 | [rpm] |
| Air supply | 2.5 | [L/min] |
| O2-supply, maximal | 2 | [L/min] |
| Temperature | 30 | [° C.] |
| O2-saturation | >40 | [%] |
| pH | 6.8 | [—] |

The bioreactor was inoculated with 100 mL inoculum. Samples of 2 mL were taken, OD₆₀₀ determined and centrifuged at 14′000 RPM. Pellet and supernatant were separated and frozen for further analysis. The biomass concentration [g/L] was calculated using the following equation:

$${OD}_{600} \times 0.45 [ g \times {L}^{- 1} \times {OD}_{600}^{- 1} ] = biomass [ \text{g/L} ] .$$

The Qbeta content in percent of the total protein content was calculated as follows, assuming, that 50% of the E coli biomass is protein:

$$Biomass [ g \times {L}^{- 1} ] / 2 = total protein [ g \times {L}^{- 1} ]$$

$$Qbeta [ g \times {L}^{- 1} ] / total protein [ g \times {L}^{- 1} ] \times 100 = Qbeta / total protein [ \% ] .$$

In the fed-batch mode, which followed the batch mode, a feeding phase was added. In the feeding phase substrate is supplied to the cells in the reactor according to a defined profile. The feed profile depends on the selected growth rate p, the yield coefficient biomass to glycerol (Y_{x/glycerol}), the volume (Vf), and the concentration of substrate in the feed (cf). substrate concentration. The feed was calculated using the following equation:

$$Feed equation$$

$$mf = ( μ / {Y}_{x / s} + m ) \qquad Vf \times Xf \times {e}^{μ t}$$

$$pump = ( mf / cf + b ) / a$$

mf=mass flow [g/h]
μ=specific growth rate [1/h]
Y_{x/glycerol}=Yield biomass to glycerol [g/g]
m=maintenance energy [g*g⁻¹*h⁻¹]
Vf=Volume at feed start
Xf=Biomass at feed start
cf=Concentration of substrate in feed [g/mL]
a+b=offset/slope of pump calibration equation

For the determination of the calibration parameters a and b, a pump calibration was carried out. In addition, the feed tube with feed bottle was clamped into the feed pump and the pump was run with 7, 14 and 21% pump performance. The pumped feed volume per time was noted. In a resulting diagram of the relation of pump performance [%] to pumped feed solution [mL/h], the slope (a) and the Y-axis section (b) was determined. On the bioreactor the parameters in Table 3 were set for fed-batch cultivation.

TABLE 3
Parameters for fed-batch cultivation in bioreactor.

| Parameter | Set point | Unit |
|---|---|---|
| Stirrer speed | 1000 | [rpm] |
| Air supply | 2.5 | [L/min] |
| O₂-supply, maximal | 2 | [L/min] |
| Temperature | 30 | [° C.] |
| O₂-saturation | >40 | [%] |
| pH | 6.8 | [—] |

After reaching a process time of approximately 7 h (end of batch) the feed pump was turned on automatically. After further 7 h cultivation, when the OD₆₀₀ reached 55-60, the feed medium (for biomass propagation) was exchanged with the induction medium R42 (for biomass propagation and induction). After 5 h feeding of R42 was stopped and the culture was harvested by centrifugation.

### Analysis of Process Parameters:

The following process parameters were routinely analysed. The pO₂, pH, temperature and stirrer speed were measured online throughout the process time. The optical density was measured offline at 600 nm. The determination of the β-galactosidase activity was performed using a β-Gal Assay Kit (Invitrogen, cat. no. K1455-01). The activity was specified as units per mL OD₆₀₀=1.0. It is defined as the quantity of Ortho-Nitrophenyl-β-D-Galactopyranosid (ONPG) in nmol, which is hydrolysed per minute and mL bacteria suspension (OD₆₀₀=1.0). The accumulated product was analysed by SDS-PAGE, the total protein content (soluble and insoluble protein) was determined and using HPLC analysis, the soluble fraction was measured. Cell disruption of E. coli was performed in lysis buffer (50 mM glucose, 25 mM tris/HCl (pH 8), 15 mM EDTA (pH 8.0)) with and ultrasonic homogeniser (Bandlin Sonoplus, HD2070). 250 μL bacteria suspension with an OD₆₀₀ of 50 were centrifuged with 14000 RPM for 10 min. The pellet was resuspended in 250 μL lysis buffer (vortex) and placed at room temperature for 5 min. Afterwards, the cells were disrupted for 20 s with ultrasonic at 10% device performance (cells on ice) and then the cell suspension was centrifuged at 14000 RPM, 10 min. The supernatant (soluble protein) was then analysed by SDS-PAGE and HPLC.

Samples before induction and at end of production (after 5 h induction) were taken from the bioreactor for analysis of Qβ formation analyzed by SDS-PAGE standardized to OD 5.0. At the end of cultivation, 1.91 of the culture was harvested. After centrifugation, the following cell pellets were obtained in three independent reactor runs: 1.) End OD₆₀₀ of 84: 194 g CWW; 2.) End OD₆₀₀ of 88: 200 g CWW; 3.) End OD₆₀₀ of 86: 201 g CWW.

The plasmid retention in run 1 and 2 was 100% at induction start and 100% at harvest. Based on comparison with a Qβ CP standard on SDS-PAGE the yield was roughly estimated to be about 5 g/l Qβ CP. HPLC analysis revealed a concentration of about 6 g/l Qβ VLP.

### Example 7

### Selection of Carbon Source and Bacterial Strain

Glucose and glycerol as carbon sources were compared. In order to test the growth behaviour of each of the strains DH20 and RB791 on these carbon sources, shake flask experiments were conducted with medium containing glucose (R27) and medium containing glycerol (R40). Both media were supplemented with 25 μg/ml kanamycin. Each culture was started with an initial OD₆₀₀ of 0.3. Induction was performed by adding 0.5% lactose. The maximum specific growth rates (μₘₐₓ) and the yield coefficients (Y_{x/s}) were determined and are listed in Table 4. RB791 grew faster on both, glucose and glycerol. In addition, the resulting yield coefficients were higher. Although glucose allowed higher maximum specific growth rates (μₘₐₓ) the yield coefficients (Y_{x/s}) was higher for glycerol.

TABLE 4
Maximum specific growth rates and the yield
coefficients of the cultivation experiments
with RB791 and DH20 on glucose and glycerol.

| | | Value after 4.5 h | | | |
|---|---|---|---|---|---|
| | | Culture Time | | Max. spec, | Yield coefficient |
| | Carbon | | Acetate | growth rate | (Y_{x/s}) biomass from |
| Strain | source | OD₆₀₀ | [g l⁻¹] | (μₘₐₓ) [h⁻¹] | substrate [g/g] |
| RB791 | glucose | 6.24 | 0.44 | 0.71 | 0.72 |
| | glycerol | 4.04 | 0.21 | 0.62 | 0.86 |
| DH20 | glucose | 2.52 | 0.42 | 0.51 | 0.71 |
| | glycerol | 2.82 | 0.25 | 0.50 | 0.81 |

### Example 8

### Determination of Optimal Temperature

The influence of temperature on product formation was investigated. Two shake flask cultures were inoculated and incubated at 30° C. and 220 rpm. After an OD₆₀₀ of 5 was reached, the cultures were induced with lactose. Subsequently, one culture was continued to be incubated at 37° C. and the other culture at 23° C. Results of the SDS-PAGE revealed that expression levels at 4 and 5 h after induction are higher in the culture induced at 37° C. Induction of the cultures for 19 h showed a higher Qβ level in the cultures induced at 23° C.

### Example 9

### Induction by Co-Feed of Lactose and Glycerol

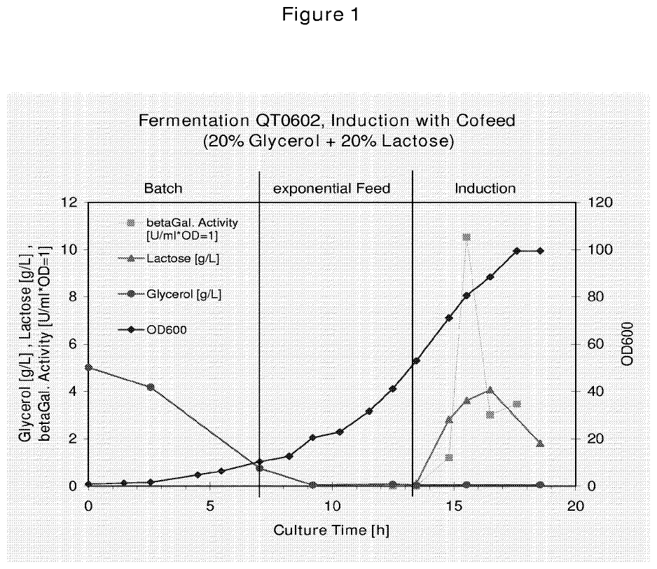

A feed solution of 20% glycerol and 20% lactose was composed (R42) and applied to fermentation as described in Example 6 at induction start. FIG. 1 provides an overview over relevant process parameters throughout the entire process time. Expression was induced at 13.5 h at an OD₆₀₀ of about 55. Upon induction, the feed pump rate was set to constant. Glycerol did not accumulate with feeding. Lactose accumulated to 4 g/l and then it started to diminish. The β-galactosidase activity rose to 10 U/ml and decreased thereafter. Compared with the previous fermentation runs a.) lactose applied as a single lactose pulse at induction start, no feeding; b.) continuous lactose feed without glycerol, the activity was with 7 U/ml higher and the maximum activity was already reached after 2 h as compared to 4 h in runs a.) and b.).

### Example 10

### Plasmid Retention

The effect of the following operating conditions on the plasmid retention was tested in the process described in Example 6: 1.) Preculture starting volume, 2.) Kanamycin in the preculture, 3.) Growth and/or induction at 37° C. vs. 30° C. The results are summarised in Table 5. Precultures were started with volumes of 5 μl out of the cell bank vial. Inoculation of a small volume allowed growth of a preculture over-night. The preculture for QT0103_F8 contained 25 mg/l kanamycin, whereas the preculture for QT0103_F7 did not contain any kanamycin. Both fermentations were operated at 30° C. and induced for 5 h. Judging from the plasmid retentions before and after 5 h induction, supplementing the preculture with kanamycin has a positive effect on plasmid retention. Plasmid retention remained at 98% before and after 5 h induction. In contrast, plasmid retentions reached only values of 80% when kanamycin was omitted from the preculture. For a subsequent run, QT0203_F7, the preculture was also started with 5 μl and grown in kanamycin containing medium. The resulting fermentation in the bioreactor was operated at 37° C. from the beginning. Operation at 37° C. had a detrimental effect on the plasmid stability. While the plasmid retention was at 99% before induction, it dropped to 0% after 5 h induction. In order to test whether a shorter preculture and thus, less generations, would improve the plasmid retention after 5 h induction, a set of precultures were started with 300 μl volume from a thawed cell bank vial and grown in kanamycin free medium. Two fermenters were operated at 30° C. for the whole run. An additional two fermenters were operated first at 30° C. for cell growth and than switched to 37° C. for the production phase. The resulting plasmid stabilities were all at 100% before and 5 h after induction.

TABLE 5
Summary of plasmid retention before and 5 h after induction obtained under different
operating conditions in terms of generations in the preculture, with and without
kanamycin in the preculture, and growth and/or induction at 37° C.

| | Preculture | | Plasmid | Plasmid | |
|---|---|---|---|---|---|
| | Starting | | retention | retention | |
| Bioreactor | Culture | Kanamycin | before | after 5 h | |
| run | Volume [μl] | in preculture | induc. [%] | induc. [%] | Remarks |
| QT0103_F8 | 5 | 25 mg/L | 98 | 98 | whole process at 30° C. |
| QT0103_F7 | 5 | no | 80 | 80 | whole process at 30° C. |
| QT0203_F7 | 5 | 25 mg/L | 99 | 0 | Bioreactor run at 37° C. |
| QT0603_F7 | 300 | no | 100 | 100 | whole process at 30° C. |
| QT0703_F8 | 300 | no | 100 | 100 | Induction at 37° C., |
| | | | | | rest of the process at 30° C. |
| QT0803_F9 | 300 | no | 100 | 100 | whole process at 30° C. |
| QT0903_F10 | 300 | no | 100 | 100 | Induction at 37° C., |
| | | | | | rest of the process at 30° C. |

### Example 11

### Variation in Time Point of Induction

In a process essentially as described in Example 6 the exponential feed profile was programmed to start 7 h after the inoculation of the bioreactor. Under standard conditions, the scheduled time for induction was at 14 h process time. In order to test the effect of variations in the time point of induction on the final cell densities, one culture was induced at 13.5 h (resulting in 6.5 h of exponential feed) and another culture at 14.5 h (resulting in 7.5 h of exponential feed). One culture induced at the regular 14 h time point served as a control (7 h of exponential feed). Results are summarised in Table 6. Cell density increased with increasing length of feeding. Judged from a linear regression analysis of the available data points for final CWW, a linear relationship appears to exist (r²=0.92).

TABLE 6
Variations in time point of induction: effect on
final cell density in terms of OD₆₀₀ and CWW.

| | Process Time | Duration of | | |
|---|---|---|---|---|
| | Point of | Exp. Feed | Final | Final |
| Reactor | Induction | Phase [h] | OD₆₀₀ | CWW [g/L] |
| F2 | 13 h 32 min | 6.5 | 83.4 | 116.5 |
| F1 | 14 h 02 min | 7.0 | 82.4 | 122.5 |
| F3 | 14 h 29 min | 7.5 | 100.4 | 141.1 |

### Example 12

### Variation in Time Point of Harvest

Harvest of the culture in a process essentially as described in Example 6 is performed manually. Under standard conditions, the scheduled time for harvest was at 19 h process time. The operation “Harvest” involves the manual ending of the bioreactor operations. In order to test the effect of variations in the time point of harvest on the final cell densities, one culture was harvested at 18.8 h (resulting in 4.8 h of induction) and another culture at 19.5 h (resulting in 5.5 h of induction). One culture harvested at the regular 19 h time point served as a control (5 h of induction). Results are summarized in Table 7. Cell density increased with increasing length of induction because the cells are still growing while induced.

TABLE 7
Variation in time point of harvest: effect on
final cell density in terms of OD600 and CWW.

| | Process Time | Length of | Final | Final |
|---|---|---|---|---|
| Reactor | Point of Harvest | Induction [h] | OD₆₀₀ | CWW [g/L] |
| F5 | 18 h 50 min | 4.8 | 91.4 | 122.4 |
| F4 | 19 h 00 min | 5.0 | 92.2 | 127.5 |
| F6 | 19 h 30 min | 5.5 | 96.0 | 132.4 |

### Example 13

### Effect of Temperature

The effect of fermentation temperature in a process essentially as described in Example 6 was investigated by running 6 fermentations at 5 different temperature setpoints. Results are summarized in Table 8. Final cell densities were sensitive to the fermentation temperature with an optimum at a temperature of 30° C.

TABLE 8
Summarized results of different temperature setpoints
on final cell density in terms of OD600 and CWW.

| | Temperature | | Final CWW |
|---|---|---|---|
| Reactor | [° C.] | Final OD₆₀₀ | [g/L] |
| F5 | 25.0 | 37.8 | 62 |
| F4 | 27.5 | 80.0 | 117 |
| F3 | 30.0 | 92.8 | 123 |
| F4 | 30.0 | 92.4 | 125 |
| F5 | 32.5 | 85.0 | 111 |
| F6 | 35.0 | 79.6 | 107 |

### Example 14

### Scaled-Up Fermentation (501)

The process described in Example 6 was scaled up to a volume of 50 l order to evaluate scale-up capability from the 2 L working volume bioreactor system to a larger volume. Key process parameters for the scaled-up process are summarized in Table 9.

TABLE 9
Process parameters of in 50 L bioreactor.

| Culture | | Time | |
|---|---|---|---|
| Step | Description | [h] | OD₆₀₀ |
| Preculture | 300 μl from cell bank vial are transferred | −11* | 5.0 |
| 1 | into 100 ml preculture medium contained in | | |
| | 500 mL shake flask and cultured for 16 h | | |
| Preculture | Calculate the required volume for transfer | −5* | 4.0 |
| 2 | in order to start with initial OD₆₀₀ of 0.3 in | | |
| | 750 ml. Tranfer calculated volume (e.g. 50 | | |
| | ml) into 750 ml preculture medium contained | | |
| | in 5000 mL shake flask | | |
| Inocula- | Pooled calculated volume (e.g. 1.4 L) is | 0 | |
| tion of | transferred into the 50 L Bioreactor. Initial | | |
| Bioreactor | volume: =40 L | | |
| Induction | The exponential feeding profile is switched | 14 | 46 |
| Start | to constant and feed is switched to induction | | |
| | feed | | |
| End of | Culture is completed after 5 h of induction | 19 | 128 |
| Culture | | | |

*Relative to the time of bioreactor inoculation.

It was necessary to have two preculture expansion steps. In the first step, the cells were expanded as established for the 2 L process (Example 6). After this step, cells were split into two 5000 ml shake flask cultures, containing 750 ml medium each. Further expansion was performed for 5 h. The cultures in the 50 L bioreactors were performed with the same time profile as in the 2 L system (Example 6). OD₆₀₀ at induction start was 46, the final OD₆₀₀ was 128. Plasmid retention was 100% before induction and 98% at the end of culture. The concentration of Qβ CP protein in the medium at the end of culture was roughly estimated 8 g/l using SDS-PAGE. The total amount of Qβ was estimated about 300 g for this reactor run.

### Example 15

### Effect of Extended Exponential Feed

The exponential feeding phase for fermentations performed according to Examples 6 or 14 was 7 h. After this time the cells reached a density for induction, which increased during induction to the targeted maximum OD₆₀₀ of around 100 to 130 as final cell density. Final OD₆₀₀, final CWW, final CDW, plasmid retention at induction start and harvest and Qβ concentration at the end of culture are determined for reactor runs performed as described in Examples 6 and 14, preferably as in Example 14, wherein the exponential feeding phase is extended to a duration up to 11 h, preferably to 10 h.

### Example 16

### Effect of Increased Feeding During Production

Example 9 demonstrates that the glycerol does not accumulate during production phase, indicating that production might be limited by the feeding rate of induction medium. Effect of extended feeding rate of induction medium on final OD₆₀₀, final CWW, final CDW, plasmid retention at induction start and harvest and Qβ concentration at the end of culture is determined in reactor runs as described in Example 6 and 14, preferably as in Example 14, wherein the feeding rate during production is increased. Alternatively or additionally, the ratio between lactose and glycerol in the feed medium shifted towards a higher glycerol and a lower lactose concentration.

### Example 17

### HPLC Analysis of Qβ CP

Qβ CP was measured with an HPLC system as follows: A sample containing Qβ CP was diluted appropriately in 1× reaction buffer (50 mM tris(hydroxymethyl)aminomethane buffer pH 8.0) containing 10 mM 1,4-Dithio-DL-threitol and incubated for 15 min at 50° C. in a thermomixer. After incubation the sample was centrifuged and the supernatant was stored at 2° C. to 10° C. until HPLC analysis. 10 to 100 μl of the sample were injected.

Qβ was quantified with a regression curve of known Qβ standards regressed to the HPLC peak area detected at 215 nm after elution from a C₄ reversed phase column, 300 Å, 5 μm, 4.6×150 mm, Vydac Inc., Hesperia, USA (Cat. No. 214TP5415) thermally equilibrated at 50° C. The flow rate through the system was 1 ml/min consisting of mobile phase A (0.12% trifluoroacetic acid in water) and mobile phase B (0.12% trifluoroacetic acid in acetonitrile) with the following gradient of phase B: 0 to 2 min constant at 40%, 2 to 8 min linear increase to 50%, 8 to 10 min constant at 50%, 10 to 10.1 min linear decrease to 40%, and 10.1 to 12 min constant at 40%.

### Example 18

### Determination of Qβ VLP by Analytical Size Exclusion Chromatography

Analysis of Qβ particles by analytical size exclusion chromatography was performed using a TskgelG5000 PW_{XL}-column (10 μm, 7.8×300 mm, TosoH Biosep; Cat.-No. 08023) equilibrated in phosphate buffered saline (20 mM Na₂HPO₄/NaH₂PO₄, 150 mM NaCl pH 7.2). Elution was performed by an isocratic gradient for 20 min at 0.8 ml/min in phosphate buffered saline. The Qbeta concentration was determined from a regression curve of known Qβ standards regressed to the HPLC peak area detected at 260 nm.

### Example 19

### Effect of Extended Exponential Feed

The exponential feeding phase for fermentations performed according to Examples 6 or 14 was 7 h. After this time the cells reached a density for induction, which increased during induction to the targeted maximum OD₆₀₀ of around 100 to 130 as final cell density. Final OD₆₀₀, final CWW, plasmid retention before induction and at harvest and Qβ concentration at the end of culture were determined for reactor runs performed as described in Examples 6 and 14, preferably as in Example 14, wherein the exponential feeding phase was extended to a duration up to 12 h. In addition, the concentration of glycerol and lactose in the induction feed were changed to 300 g/L and 100 g/L respectively. The results are summarized in Table 10.

TABLE 10
OD₆₀₀ and CWW at the end of cultivations, Plasmid Retention
before induction and at the end of cultivations as well
as the peak oxygen mass flow. The cultivations were conducted
with different duration of exponential feeding.

| Duration | | | | | |
|---|---|---|---|---|---|
| Exponential | | | Plasmid Retention [%] | | Peak Oxygen |
| Feeding | OD₆₀₀ | CWW | before | end of | Mass Flow |
| [h] | [—] | [g/L] | induction | cultivation | [vvm] |
| 7 | 86 | 122 | 100 | 99 | 0.2 |
| 8 | 112 | 184 | 99 | 98 | 0.4 |
| 9 | 136 | 217 | 100 | 98 | 0.8 |
| 10 | 164 | 228 | 99 | 98 | 1.5 |
| 11 | 200 | 262 | 100 | 97 | >4.5 |
| 12 | 90 | 186 | 99 | 100 | >4.5 |

According to LDS-PAGE analysis, the specific Qbeta concentration of all cultivations except for the cultivation with 12 h exponential feeding was the same. An optimum regarding absolute Qbeta yield and oxygen consumption was found for 9.5 h exponential feeding. Therefore, the process is preferably run with 9.5 h exponential feeding phase.

### Example 20

### Scaled Up Fermentation (501)

The process described in Example 6 and with 9.5 h exponential feeding phase with 300 g/L glycerol and 100 g/L lactose as described in Example 19 was scaled up to a volume of 50 L in order to evaluate scale-up capability from the 2 L working volume bioreactor system to a larger volume. Key process parameters for the scaled up process are summarized in Table 11.

TABLE 11
Process parameters on the 50 L scale

| | | Time |
|---|---|---|
| Culture Step | Description | [h] |
| Preculture | 200 μl from cell bank vial were transferred | −18* |
| | into 800 ml preculture medium contained in | |
| | 3000 mL shake flask and cultured for 18 h | |
| | (2 flasks) | |
| Inoculation | Pooled total volume (approx. 1.6 L) was | 0 |
| of Bioreactor | transferred into the 50 L Bioreactor. Initial | |
| | volume: =35 L | |
| Induction | The exponential feeding profile was | 16.5 |
| Start | switched to constant and feed was switched | |
| | to induction feed | |
| End of | Culture was completed after 5 h of | 21.5 |
| Culture | induction | |

*Relative to the time of bioreactor inoculation.

It was necessary to change the preculture procedure in order to inoculate the larger reactor with approximately the same cell density. The cultures in the 50 L bioreactors were performed with the time profile optimised for the 2 L scale as described in Example 19. The final cell wet weight for six cultivations was 188 g/L±9. Plasmid retention was 97.3%±1.4 at the end of culture. The concentration of Qβ CP protein in the medium at the end of culture was determined by C₄ reversed phase HPLC (Example 17) to 10.8 g/L±0.3. The total amount of Qβ CP was 540 g for one 50 L run. The crude extract of approximately two times concentrated biomass was analysed for Qβ CP and Qβ VLP (Example 18). The concentration of Qβ CP was 19.1 g/L±0.4 (C₄ reversed phase HPLC), the concentration of Qβ VLP was 18.8 g/L±1.1. Therefore, the VLP-yield of the fermentation process is estimated to approximately 9-11 g/l fermentation broth at the time of harvest.

1. A process for expression of a recombinant capsid protein of a RNA bacteriophage being capable of forming a virus-like particle (VLP) by self-assembly, said process comprising the steps of:
a.) introducing an expression plasmid into a bacterial host, wherein said expression plasmid comprises an expression construct, wherein said expression construct comprises (i) a first nucleotide sequence encoding said recombinant capsid protein, or mutant or fragment thereof, and (ii) a promoter being inducible by lactose;
b.) cultivating said bacterial host in a medium comprising a major carbon source; wherein said cultivating is performed in batch culture and under conditions under which said promoter is repressed by lacI, wherein said lacI is overexpressed by said bacterial host;
c.) feeding said batch culture with said major carbon source; and
d.) inducing said promoter with an inducer, wherein said feeding of said batch culture with said major carbon source is continued.

2. (canceled)

3. The process of claim 1, wherein said bacteriophage is selected from the group consisting of:
a.) bacteriophage Qβ;
b.) bacteriophage AP205;
c.) bacteriophage fr;
d.) bacteriophage GA;
e.) bacteriophage SP;
f.) bacteriophage MS2;
g.) bacteriophage M11;
h.) bacteriophage MX1;
i.) bacteriophage NL95;
j.) bacteriophage f2;
k.) bacteriophage PP7 and
l.) bacteriophage R17.

4. The process of claim 1, wherein said RNA bacteriophage is Qβ.

5. The process of claim 1, wherein said recombinant capsid protein has the amino acid sequence of SEQ ID NO:5.

6. The process of claim 1, wherein said expression construct comprises a first stop codon and a second stop codon, wherein said first stop codon is located directly 3′ of said first nucleotide sequence and wherein said second stop codon is located directly 3′ of said first stop codon, and wherein at least one of said first or second stop codon is TAA.

7. The process of claim 1, wherein said expression construct comprises a first nucleotide sequence and a second nucleotide sequence, wherein said first nucleotide sequence is encoding Qβ coat protein (CP), and wherein said second nucleotide sequence is encoding the Qβ A1 protein or a mutant or fragment thereof, and wherein said first and said second nucleotide sequence are separated by exactly one sequence stretch comprising at least one TAA stop codon.

8. The process of claim 1, wherein said expression construct comprises the nucleotide sequence of SEQ ID NO:6.

9. The process of claim 1, wherein said expression plasmid comprises the nucleotide sequence of SEQ ID NO:1.

10. (canceled)

11. The process of claim 1, wherein said promoter is selected from the group consisting of the
a.) tac promoter;
b.) trc promoter;
c.) tic promoter;
d.) lac promoter;
e.) lacUV5 promoter;
f.) P_{syn} promoter;
g.) lpp^{a} promoter;
h.) lpp-lac promoter;
i.) T7-lac promoter;
j.) T3-lac promoter;
k.) T5-lac promoter; and
l.) a promoter having at least 50% sequence homology to SEQ ID NO:2.

12. The process of claim 1, wherein said promoter comprises the nucleotide sequence of SEQ ID NO:2.

13. The process of claim 1, wherein said major carbon source is glycerol.

14. The process of claim 1, wherein said feeding of said batch culture is performed with a flow rate, wherein said flow rate increases with an exponential coefficient μ.

15. The process of claim 1, wherein said inducing of said promoter is performed by co-feeding said batch culture with said inducer and said major carbon source at a constant flow rate.

16. The process of claim 1, wherein said inducing of said promoter is performed by co-feeding said batch culture with said inducer and said major carbon source at an increasing flow rate.

17. The process of claim 15, wherein said inducer is lactose and wherein said lactose and said major carbon source are co-fed to said batch culture in a ratio of about 2:1 to 1:4 (w/w).

18. The process of claim 1, wherein said inducer is IPTG and wherein the concentration of said IPTG in said medium is 0.001 to 5 mM.

19. (canceled)

20. The process of claim 1, wherein said lacI is overexpressed by said bacterial host, wherein said overexpression is caused by lacI^{q} or lacQ1.

21. (canceled)

22. (canceled)

23. The process of claim 1, wherein said inducer is lactose and wherein said bacterial host comprises β-galactosidase activity.

24. The process of claim 1, wherein said cultivating and said feeding of said batch culture and said inducing of said promoter is performed at a temperature which is below the optimal growth temperature of said bacterial host.

25. The process of claim 1 wherein:
a.) said major carbon source is glycerol;
b.) said feeding of said batch culture is performed with a flow rate, wherein said flow rate increases with an exponential coefficient μ, and wherein said exponential coefficient μ is below μₘₐₓ;
c.) said inducer is lactose;
d.) and said lactose and said major carbon source are co-fed to said batch culture in a ratio of 2:1 to 1:4 (w/w);
e.) said bacterial host is E. coli RB791; and
f.) said cultivating and feeding of said batch culture and said inducing of said promoter is performed at a temperature of about 30° C.

26. (canceled)

27. The process of claim 1, wherein throughout steps b.) to d.) of said process oxygen is supplied to said bacterial host, wherein said oxygen supply is effected such that the partial pressure of oxygen in the medium (pO₂) is at least about 40%.


SEQUENCE LISTING (40 sequences)

SEQ ID NO: 1 - DNA, 5579 bp, synthetic construct
  misc_feature 1..5579: note = plasmid
  source 1..5579: mol_type = other DNA
ggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattctaaggaggaaaaaaaaatggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatatgctgacgtgaccttttcgttcacgcagtatagtaccgatgaggaacgagcttttgttcgtacagagcttgctgctctgctcgctagtcctctgctgatcgatgctattgatcagctgaacccagcgtattaatgactgctcattgccggtggtggctcagggtcaaaacccgatccggttattccggatccaccgattgatccgccgccagggacaggtaagtatacctgtcccttcgcaatttggtccctagaggaggtttacgagcctcctactaagaaccgaccgtggcctatctataatgctgttgaactccagcctcgcgaatttgatgttgccctcaaagatcttttgggcaatacaaagtggcgtgattgggattctcggcttagttataccacgttccgcggttgccgtggcaatggttatattgaccttgatgcgacttatcttgctactgatcaggctatgcgtgatcagaagtatgatattcgcgagggcaagaaacctggtgctttcggtaacattgagcgattcatttatcttaagtcgataaatgcttattgctctcttagcgatattgcggcctatcacgccgatggcgtgatagttggcttttggcgcgatccatccagtggtggtgccataccgtttgacttcactaagtttgataagactaaatgtcctattcaagccgtgatagtcgttcctcgtgcttagtaactaaggatgaaatgcatgtctaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctagagccacgttgtgtctcaaaatctctgatgttacattgcacaagataaaaatatatcatcatgaacaataaaactgtctgcttacataaacagtaatacaaggagtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaacgcgtgaccaagtttactcatatgtactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagctcccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtat

SEQ ID NO: 2 - DNA, 245 bp, synthetic construct
  misc_feature 1..245: note = promoter sequence
  source 1..245: mol_type = other DNA
cgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacag

SEQ ID NO: 3 - DNA, 19 bp, synthetic construct
  misc_feature 1..19: note = Shine-Dalgarno Sequence
  source 1..19: mol_type = other DNA
taaggaggaaaaaaaaatg

SEQ ID NO: 4 - DNA, 18 bp, synthetic construct
  misc_feature 1..18: note = Shine-Dalgarno Sequence
  source 1..18: mol_type = other DNA
aggaggtaaaaaacgatg

SEQ ID NO: 5 - protein, 133 aa, Bacteriophage Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 6 - DNA, 1017 bp, synthetic construct
  misc_feature 1..1017: note = Expression construct
  source 1..1017: mol_type = other DNA
atggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatatgctgacgtgaccttttcgttcacgcagtatagtaccgatgaggaacgagcttttgttcgtacagagcttgctgctctgctcgctagtcctctgctgatcgatgctattgatcagctgaacccagcgtattaatgactgctcattgccggtggtggctcagggtcaaaacccgatccggttattccggatccaccgattgatccgccgccagggacaggtaagtatacctgtcccttcgcaatttggtccctagaggaggtttacgagcctcctactaagaaccgaccgtggcctatctataatgctgttgaactccagcctcgcgaatttgatgttgccctcaaagatcttttgggcaatacaaagtggcgtgattgggattctcggcttagttataccacgttccgcggttgccgtggcaatggttatattgaccttgatgcgacttatcttgctactgatcaggctatgcgtgatcagaagtatgatattcgcgagggcaagaaacctggtgctttcggtaacattgagcgattcatttatcttaagtcgataaatgcttattgctctcttagcgatattgcggcctatcacgccgatggcgtgatagttggcttttggcgcgatccatccagtggtggtgccataccgtttgacttcactaagtttgataagactaaatgtcctattcaagccgtgatagtcgttcctcgtgcttagtaactaaggatgaaatgcatgtctaag

SEQ ID NO: 7 - protein, 132 aa, Bacteriophage Qbeta
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 8 - protein, 132 aa, Bacteriophage Qbeta
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 9 - protein, 132 aa, Bacteriophage Qb
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 10 - protein, 132 aa, Bacteriophage Qbeta
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 11 - protein, 132 aa, Bacteriophage Qbeta
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 12 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 13 - protein, 131 aa, Bacteriophage AP205
MANKTMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 14 - protein, 131 aa, Bacteriophage AP205
MANKPMQPITSTADKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 15 - protein, 329 aa, Bacteriophage Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 16 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 17 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 18 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 19 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 20 - protein, 329 aa, Bacteriophage
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 21 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 22 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 23 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 24 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 25 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 26 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 27 - DNA, 4586 bp, synthetic construct
  misc_feature 1..4586: note = Cloning Vector
  source 1..4586: mol_type = other DNA
ttctgtttcctgtgtgaaattgttatccgctcacaattccacacattatacgagccgatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgataagctccggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagaagcttggctgcaggtcgacggatccccgggaa

SEQ ID NO: 28 - DNA, 425 bp, synthetic construct
  misc_feature 1..425: note = Terminator Sequence
  source 1..425: mol_type = other DNA
ctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctt

SEQ ID NO: 29 - DNA, 816 bp, synthetic construct
  misc_feature 1..816: note = Resistance gene
  source 1..816: mol_type = other DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcat

SEQ ID NO: 30 - DNA, 4963 bp, synthetic construct
  misc_feature 1..4963: note = Plasmid
  source 1..4963: mol_type = other DNA
ggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattctaaggaggaaaaaaaaatggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaatgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctagagccacgttgtgtctcaaaatctctgatgttacattgcacaagataaaaatatatcatcatgaacaataaaactgtctgcttacataaacagtaatacaaggagtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaacgcgtgaccaagtttactcatatgtactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagctcccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtat

SEQ ID NO: 31-32 - skipped (2 entries)

SEQ ID NO: 33 - DNA, 4525 bp, synthetic construct
  misc_feature 1..4525: note = Plasmid
  source 1..4525: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcagacatgcatttcatccttagttactaagcacgaggaacgactatcacggcttgaataggacatttagtcttatcaaacttagtgaagtcaaacggtatggcaccaccactggatggatcgcgccaaaagccaactatcacgccatcggcgtgataggccgcaatatcgctaagagagcaataagcatttatcgacttaagataaatgaatcgctcaatgttaccgaaagcaccaggtttcttgccctcgcgaatatcatacttctgatcacgcatagcctgatcagtagcaagataagtcgcatcaaggtcaatataaccattgccacggcaaccgcggaacgtggtataactaagccgagaatcccaatcacgccactttgtattgcccaaaagatctttgagggcaacatcaaattcgcgaggctggagttcaacagcattatagataggccacggtcggttcttagtaggaggctcgtaaacctcctctagggaccaaattgcgaagggacaggtatacttacctgtccctggcggcggatcaatcggtggatccggaataaccggatcgggttttgaccctgagccaccaccggcaatgagcagtcattaatacgctgggttcagctgatcaatagcatcgatcagcagaggactagcgagcagagcagcaagctctgtacgaacaaaagctcgttcctcatcggtactatactgcgtgaacgaaaaggtcacgtcagcatatgcctggcgagtaacggatgggtcacaagaaccgtttgcagtgcaagcggtcgggttctggatcttaacctggaccttgtagttcttacgattgcgagaaggctgagataccgaaacggtaacacgcttctccagcgcaggaactgcacccgcttgtgaaagcgaggcaacgccgttagtgggatttaccccacgcggattgaggaccagagtttgttttccatctttcccgatgttacctaaagtaacagtctctaattttgccatcgttttttacctccttctagagtcattatggttttgccatacatcagtatggtgtagcagcacttattataatctttattgcctcttaaaacttaatccacatcaaaactcaaatacttttaaccccagcgtcctgtaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 34 - DNA, 56 bp, synthetic construct
  misc_feature 1..56: note = Primer Sequence
  source 1..56: mol_type = other DNA
gcgcgcgaattcaggaggtaaaaaacgatggcaaaattagagactgttactttagg

SEQ ID NO: 35 - DNA, 33 bp, synthetic construct
  misc_feature 1..33: note = Primer Sequence
  source 1..33: mol_type = other DNA
gcatgcaagcttagacatgcatttcatccttag

SEQ ID NO: 36 - DNA, 57 bp, synthetic construct
  misc_feature 1..57: note = Primer Sequence
  source 1..57: mol_type = other DNA
gcgcgcgaattctaaggaggaaaaaaaaatggcaaaattagagactgttactttagg

SEQ ID NO: 37 - DNA, 3914 bp, synthetic construct
  misc_feature 1..3914: note = Cloning Vector
  source 1..3914: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccccggatccgtcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatccggggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 38 - DNA, 57 bp, synthetic construct
  misc_feature 1..57: note = Primer sequence
  source 1..57: mol_type = other DNA
gcgcgcgaattctaaggaggaaaaaaaaatggcaaataagccaatgcaaccgatcac

SEQ ID NO: 39 - DNA, 44 bp, synthetic construct
  misc_feature 1..44: note = Primer Sequence
  source 1..44: mol_type = other DNA
gcatgcaagcttcattaagcagtagtatcagacgatacgatagc

SEQ ID NO: 40 - DNA, 4962 bp, synthetic construct
  misc_feature 1..4962: note = Expression Construct
  source 1..4962: mol_type = other DNA
ggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattcaggaggtaaaaaacgatggcaaataagccaatgcaaccgatcacatctacagcaaataaaattgtgtggtcggatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaatgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctagagccacgttgtgtctcaaaatctctgatgttacattgcacaagataaaaatatatcatcatgaacaataaaactgtctgcttacataaacagtaatacaaggagtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaacgcgtgaccaagtttactcatatgtactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagctcccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtat